(19) United States
(12) Patent Application Publication
(10) Pub. No.: US 2025/0342919 A1
(43) Pub. Date: Nov. 6, 2025

(54) APPARATUS AND METHOD FOR CLASSIFYING A USER TO A COHORT OF RETROSPECTIVE USERS

(71) Applicant: Anumana, Inc., Cambridge, MA (US)

(72) Inventors: Rakesh Barve, Bengaluru (IN); Samir Awasthi, Boston, MA (US); Arjun Puranik, San Jose, CA (US); Uddeshya Upadhyay, Bengaluru (IN); Akash Anand, Bhagalpur (IN); Suthirth Vaidya, Bengaluru (IN)

(73) Assignee: Anumana, Inc., Cambridge, MA (US)

(21) Appl. No.: 18/652,921

(22) Filed: May 2, 2024

(51) Int. Cl. G16H 10/60

(52) U.S. Cl. CPC G16H 10/60

(57) ABSTRACT

An apparatus and method for classifying a user to a cohort of retrospective users is disclosed. The apparatus includes at least a processor and a computer-readable storage medium communicatively connected to the at least a processor, wherein the computer-readable storage medium contains instructions configuring the at least processor to receive user data of a user, generate a vector embedding of the user data, generate a query input, generate a plurality of cohorts of retrospective users using cohort data extracted from a cohort database based on the query input, wherein generating the plurality of cohorts includes generating a set of vector embeddings of the cohort data, classify, based on the vector embedding and the set of vector embeddings, the user data to at least a cohort of the plurality of cohorts of the retrospective users, and output the at least a cohort through a user interface.

### FIELD OF THE INVENTION

The present invention generally relates to the field of user classification. In particular, the present invention is directed to an apparatus and method for classifying a user to a cohort of retrospective users.

### BACKGROUND

There exists a significant challenge in accurately classifying a user's medical experiences by aligning them with historical data from past patients. The complexity arises from the vast diversity of patient histories, the subtleties of individual medical conditions, and the dynamic nature of healthcare data. Misclassification can lead to inaccurate analyses, which might affect treatment plans and patient outcomes. Therefore, there's a pressing need to optimize this classification process, ensuring that current patient data is precisely matched with relevant historical records. By enhancing this alignment, we can improve the accuracy of predictive analytics, tailor treatments more effectively, and ultimately elevate the standard of patient care.

### SUMMARY OF THE DISCLOSURE

In an aspect, an apparatus for classifying a user to a cohort of retrospective users is disclosed. The apparatus includes at least a processor and a computer-readable storage medium communicatively connected to the at least a processor, wherein the computer-readable storage medium contains instructions configuring the at least processor to receive user data of a user, wherein the user data includes medical data. The computer-readable storage medium further contains instructions configuring the at least a processor to generate a vector embedding of the user data. The computer-readable storage medium further contains instructions configuring the at least a processor to generate a query input. The computer-readable storage medium further contains instructions configuring the at least a processor to generate a plurality of cohorts of retrospective users using cohort data extracted from a cohort database based on the query input, wherein generating the plurality of cohorts includes generating a set of vector embeddings of the cohort data. The computer-readable storage medium further contains instructions configuring the at least a processor to classify, based on the vector embedding and the set of vector embeddings, the user data to at least a cohort of the plurality of cohorts of the retrospective users. The computer-readable storage medium further contains instructions configuring the at least a processor to output the at least a cohort through a user interface.

In another aspect, a method for classifying a user to a cohort of retrospective users is described. The method includes receiving, by a computing device, user data of a user, wherein the user data includes medical data. The method further includes generating, by the computing device, a vector embedding of the user data. The method further includes generating, by the computing device, a query input. The method further includes generating, by the computing device, a plurality of cohorts of retrospective users using cohort data extracted from a cohort database based on the query input, wherein generating the plurality of cohorts includes generating a set of vector embeddings of the cohort data. The method further includes classifying, by the computing device, based on the vector embedding and the set of vector embeddings, the user data to at least a cohort of the plurality of cohorts of the retrospective users. The method further includes outputting, by the computing device, the at least a cohort through a user interface.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is an illustration of an exemplary apparatus for classifying a user to a cohort of retrospective users;

FIG. 2 is an illustration of an exemplary user interface;

FIG. 3 is an illustration of an exemplary user interface;

FIG. 4 is an illustration of an exemplary user interface;

FIG. 5 is an illustration of an exemplary user interface;

FIG. 6 is an illustration of an exemplary user interface;

FIG. 7 is an illustration of an exemplary user interface;

FIG. 8 is an illustration of an exemplary user interface;

FIG. 9 is an illustration of an exemplary user interface;

FIGS. 10A-F illustrate exemplary embodiments of a plurality of models used to generate each of representation of the plurality of representations;

FIG. 11 is an illustration of an exemplary user interface;

FIG. 12 is a box diagram of an exemplary machine learning model;

FIG. 13 is a diagram of an exemplary neural network;

FIG. 14 is a diagram of an exemplary neural network node;

FIG. 15 is a diagram of an exemplary fuzzy inference system;

FIG. 16 is a flow diagram depicting an exemplary embodiment of a method for classifying a user to a cohort of retrospective users; and

FIG. 17 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to apparatuses and methods for classifying a user to a cohort of retrospective users. By accurately classifying patients into specific cohorts based on their comprehensive medical profiles, healthcare providers can offer more personalized treatment plans. This targeted approach ensures that treatments are optimized for the specific characteristics and needs of each patient group, potentially increasing efficacy and reducing adverse effects.

Aspects of the present disclosure can be used to predict outcomes for individual patients based on historical data from similar patient cohorts. For instance, if a patient's data aligns closely with a cohort that has a known trajectory or response to treatment, healthcare providers can use this information to make informed predictions about the patient's future health status or response to certain therapies.

Aspects of the present disclosure can also be used to streamline clinical trial design by identifying patient cohorts with specific characteristics, making it easier to recruit suitable candidates for trials investigating particular conditions or treatments.

Exemplary embodiments illustrating aspects of the present disclosure are described below in the context of several specific examples.

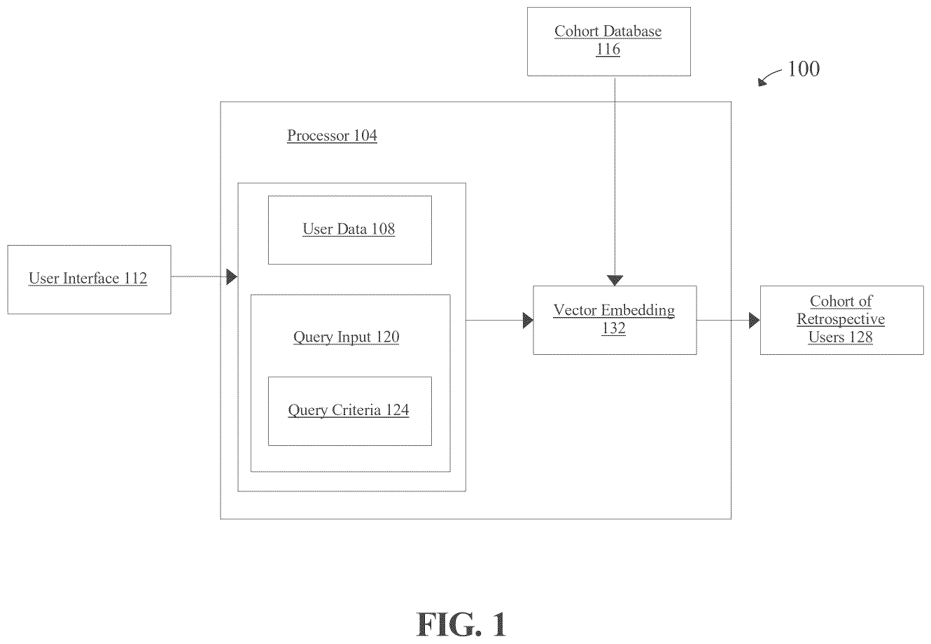

Referring now to FIG. 1, an exemplary embodiment of an apparatus 100 for classifying a user to a cohort of retrospective users is illustrated. Apparatus 100 includes a processor 104 communicatively connected to a memory. As used in this disclosure, “communicatively connected” means connected by way of a connection, attachment or linkage between two or more relata which allows for reception and/or transmittance of information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

Further referring to FIG. 1, processor 104 may include any computing device as described in this disclosure, including without limitation a microcontroller, microprocessor, digital signal processor (DSP) and/or system on a chip (SoC) as described in this disclosure. Processor 104 may include, be included in, and/or communicate with a mobile device such as a mobile telephone or smartphone. Processor 104 may include a single computing device operating independently, or may include two or more computing device operating in concert, in parallel, sequentially or the like; two or more computing devices may be included together in a single computing device or in two or more computing devices. Processor 104 may interface or communicate with one or more additional devices as described below in further detail via a network interface device. Network interface device may be utilized for connecting processor 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Processor 104 may include but is not limited to, for example, a computing device or cluster of computing devices in a first location and a second computing device or cluster of computing devices in a second location. Processor 104 may include one or more computing devices dedicated to data storage, security, distribution of traffic for load balancing, and the like. Processor 104 may distribute one or more computing tasks as described below across a plurality of computing devices of computing device, which may operate in parallel, in series, redundantly, or in any other manner used for distribution of tasks or memory between computing devices. processor 104 may be implemented, as a non-limiting example, using a “shared nothing” architecture.

With continued reference to FIG. 1, processor 104 may be designed and/or configured to perform any method, method step, or sequence of method steps in any embodiment described in this disclosure, in any order and with any degree of repetition. For instance, processor 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Processor 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, processor 104 is configured to receive user data 108. “User data,” as used herein is data related to a person. User data 108 may include medical data. “Medical data,” for the purposes of this disclosure, is user data that is related to the treatment, diagnosis, or monitoring of illnesses, diseases, disorders, risk factors, or injuries. For example, a person may refer to a patient seeking medical attention and/or advice. User data 108 may include ECG (electrocardiogram) data. ECG data may include digital ECG data and/or analog ECG data. As used in the current disclosure, “digital ECG data” refers to the digital representation of the electrical activity of the heart recorded over time. As used in the current disclosure, “analog ECG data” refers to an analog representation of the electrical activity of the heart recorded over time. ECG data may include a plurality of ECG signals represented in a digital or analog format. As used in the current disclosure, a “format” refers to a method of representing information or data using continuous and continuously variable physical quantities, such as electrical voltage. Electrical activity may be depicted using electrocardiogram (ECG) signals. As used in the current disclosure, a “electrocardiogram signal” is a signal representative of electrical activity of heart. The ECG signal may consist of several distinct waves and intervals, each representing a different phase of the cardiac cycle. These waves may include the P-wave, QRS complex, T wave, U wave, and the like. The P-wave may represent atrial depolarization (contraction) as the electrical impulse spreads through the atria. The QRS complex may represent ventricular depolarization (contraction) as the electrical impulse spreads through the ventricles. The QRS complex may include three waves: Q wave, R wave, and S wave. The T-wave may represent ventricular repolarization (recovery) as the ventricles prepare for the next contraction. The U-wave may sometimes be present after the T wave, it represents repolarization of the Purkinje fibers. The intervals between these waves provide information about the duration and regularity of various phases of the cardiac cycle. The ECG signal can help diagnose various heart conditions, such as arrhythmias, myocardial infarction (heart attack), conduction abnormalities, and electrolyte imbalances.

Still referring to FIG. 1, in some embodiments, processor 104 may receive ECG data in the form of an ECG printout and be configured to covert to the printout to a digital format as disclosed in Non-provisional application Ser. No. 18/599,435 (Attorney Docket No. 1518-115USU1) filed on Mar. 3, 2024 and entitled “AN APPARATUS AND METHOD FOR GENERATING A QUALITY DIAGNOSTIC OF ECG (ELECTROCARDIOGRAM) DATA,” the entirety of which is incorporated herein by reference. An “ECG printout,” as used herein, is a graphical representation of the electrical activity of the heart recorded over a period of time. As disclosed in Ser. No. 18/599,435, processor 104 may receive ECG data, extract a plurality of ECG parameters from the ECG data and convert the ECG data to one or more digitized ECG signals.

Still referring to FIG. 1, user data 108 may include electronic health records. An “electronic health record (EHR),” as used herein, is an electronic version of a user's medical history. An EHR may be maintained by a provider, such as a physician, over time, and may include all of the key administrative clinical data relevant the user's care under a particular provider, including demographics, progress notes, problems, medications, vital signs, past medical history, immunizations, laboratory data and radiology reports. For example, EHR demographics may include age, gender, socioeconomic status, geographic location, marital status, language/communication needs and the like.

Still referring to FIG. 1, user data 108 may include a user profile. A “user profile,” as used herein is a data structure containing racial, physical or personal attributes, and/or identification of a user. A user profile may contain data disclosed or missing from an EHR. A user profile may contain data updating or commenting on information in the EHR. The user profile may contain data received from a user and not a medical provider or EHR. For example, the user profile may include allergies, medical history, family medical history, smoking status, exercise/dietary habits, pharmacy information, legal documents, such as healthcare proxy, patient identification, contact information, and current symptoms expressed by user, such as indications of pain level, areas of pain, and the like. The EHR and user profile may contain unique identifiers correlated to the user within the healthcare system. In some embodiments, EHR and/or user profile may include physical attributes of a user. A “physical attribute,” as used herein, includes any characteristic or feature of an individual's outward appearance. For example, physical attributes may include, but are not limited to, height, weight, hair color, eye color, skin tone, facial features, and body shape. In some embodiments, EHR and/or user profile may include racial attributes of a user. A “racial attribute,” as used herein, includes any characteristic or feature of an individual associated with a racial group. Racial attributes may include physical descriptions. For example, skin color can influence the presentation of symptoms or conditions, and knowing a patient's racial background may alert a healthcare provider to consider certain genetic conditions more common in that population. Racial attributes may include genetic traits, as some genetic traits are more prevalent in certain racial groups. This may include certain genetic markers, blood types, or predispositions to specific health conditions.

Still referring to FIG. 1, in some embodiments processor 104 may generate a user profile based on data received using any method described herein. For example, processor 104 may use an optical character recognition process, language processing algorithm, and/or a machine-learning model such as a classifier to index or categorize data received to elements of a user profile. Categories of the user profile may include aspects a disclosed above, such as smoking status, contact information, and the like. Categories and/or a template user profile may be received from a third party, such a healthcare physician, or an apparatus 100 operator to indicate data to be filled in.

Still referring to FIG. 1, processor 104 may receive user data 108 as input through a user interface 112. A “user interface,” as used herein, is a means by which a user and a computer system interact; for example, through the use of input devices and software. A user interface 112 may include a graphical user interface (GUI), command line interface (CLI), menu-driven user interface, touch user interface, voice user interface (VUI), form-based user interface 112, any combination thereof, and the like. A user interface 112 may include a smartphone, smart tablet, desktop, or laptop operated by the user. In an embodiment, the user interface 112 may include a graphical user interface. A “graphical user interface (GUI),” as used herein, is a graphical form of user interface that allows users to interact with electronic devices. In some embodiments, GUI may include icons, menus, other visual indicators, or representations (graphics), audio indicators such as primary notation, and display information and related user controls. A menu may contain a list of choices and may allow users to select one from them. A menu bar may be displayed horizontally across the screen such as pull-down menu. When any option is clicked in this menu, then the pulldown menu may appear. A menu may include a context menu that appears only when the user performs a specific action. An example of this is pressing the right mouse button. When this is done, a menu may appear under the cursor. Files, programs, web pages and the like may be represented using a small picture in a graphical user interface. For example, links to decentralized platforms as described in this disclosure may be incorporated using icons. Using an icon may be a fast way to open documents, run programs etc. because clicking on them yields instant access. Information contained in user interface 112 may be directly influenced using graphical control elements such as widgets. A “widget,” as used herein, is a user control element that allows a user to control and change the appearance of elements in the user interface. In this context a widget may refer to a generic GUI element such as a check box, button, or scroll bar to an instance of that element, or to a customized collection of such elements used for a specific function or application (such as a dialog box for users to customize their computer screen appearances). User interface 112 controls may include software components that a user interacts with through direct manipulation to read or edit information displayed through user interface 112. Widgets may be used to display lists of related items, navigate the system using links, tabs, and manipulate data using check boxes, radio boxes, and the like. Additionally or alternatively the user interface 112 may integrate a chatbot to receive user data 108. For example, the chatbot may greet a patient and ask for data related to filling out a user profile such as basic identification details like name and date of birth. The chatbot may guide the patient through various sections of the form/user profile, asking straightforward questions about medical history, insurance, current medications, allergies, lifestyle habits, pain assessment, and the like.

Still referring to FIG. 1, processor 104 may receive user data 108 from a user database. A “user database,” as used herein, is data structure contacting data related to the user. Databases as described herein may be implemented, without limitation, as a relational database, a key-value retrieval database such as a NOSQL database, or any other format or structure for use as a database that a person skilled in the art would recognize as suitable upon review of the entirety of this disclosure. Databases may alternatively or additionally be implemented using a distributed data storage protocol and/or data structure, such as a distributed hash table or the like. Databases may include a plurality of data entries and/or records as described above. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure. In some embodiments, the user database may be populated by the chatbot, or from inputs received through the user interface 112.

Still referring to FIG. 1, user data 108 may include one or more symptoms. A “symptom,” as use herein is a subjective indication of a disease, disorder, or abnormal condition that is experienced by an individual and is observable or perceivable by the affected person or others. Symptoms may include indications of pain, discomfort, changes in bodily functions, sensations, or emotions. Examples of symptoms include fever, cough, headache, fatigue, nausea, dizziness, and shortness of breath, among many others. Symptoms may be assessed from ECG data, the EHR and/or the user profile. Symptoms may be received from a third-party input into the user interface 112. For example, a health professional may examine a user and input symptoms observed.

Still referring to FIG. 1, processor 104 may be configured to generate abnormality datum. In some embodiments, abnormality datum may be generated as a function of image based user data 108, such as ECG data. As used herein, an “abnormality datum” is a data structure describing a difference between a signal and a typical signal of a healthy individual. In some embodiments, abnormality datum may be determined as a function of signal metric and/or signal metric position. As used herein, a “signal metric position” is a data structure describing the position of a signal metric relative to that of one or more members of a population. As a non-limiting example, a signal metric position may indicate that a subject's PR interval is higher than 55% of a population. In a non-limiting example, apparatus 100 may generate abnormality datum based on signal metric being above or below a threshold. Such a threshold may be determined as a function of information about a subject associated with signal, such as age, sex, medical history, and the like. In another non-limiting example, apparatus 100 may generate abnormality datum based on signal metric position being above or below a threshold. In a non-limiting example, apparatus 100 may generate abnormality datum if signal metric position indicates that signal metric is in the top 5% of a population.

Still referring to FIG. 1, in some embodiments, apparatus 100 may generate abnormality datum using an abnormality datum machine learning model. Abnormality datum machine learning model may be trained using a supervised learning algorithm. Abnormality datum machine learning model may be trained on a training dataset including example images, signal metrics, and/or calibration data, associated with example anomaly data. Such a training dataset may be obtained by, for example, gathering diagnoses of retrospective users, as described further below, and associating those diagnoses with images of ECG data of those subjects. Once abnormality datum machine learning model is trained, it may be used to determine anomaly data. Apparatus 100 may input ECG image, signal metric, and/or calibration datum into abnormality datum machine learning model, and apparatus 100 may receive abnormality datum from the model.

Still referring to FIG. 1, in some embodiments, apparatus 100 may generate abnormality datum confidence score. In some embodiments, abnormality datum machine learning model may output abnormality datum confidence score in addition to its other outputs. As used herein, a “confidence score” is a degree of confidence that an associated datum is accurate. As used herein, an “abnormality datum confidence score” is a degree of confidence that an abnormality datum is accurate. In some embodiments, a confidence score may be determined as a function of a machine learning model, such as abnormality datum machine learning model. Confidence scores may be used to predict how likely a model output is to be accurate. For example, in some classifiers, numerical values are calculated, and a cutoff value is used to determine which category the input fits into. In this example, the numerical value may be used to determine a certainty score based on how closely it fits into a class and/or how close to a decision boundary it is. In another example, in clustering algorithms, certainty scores may be calculated based on how closely an input fits into a cluster. In some embodiments, abnormality datum is generated without the use of abnormality datum machine learning model, and abnormality datum confidence score is generated using other methods. For example, where abnormality datum is determined as a function of a comparison between signal metric and a threshold, abnormality datum may be determined as a function of the distance between signal metric and the threshold. The abnormality datum may be included a parameter or search criteria for classifying a user to a cohort ss described further below. Both the abnormality datum and abnormality datum confidence score may be displayed through a user interface 112 as described further below.

Still referring to FIG. 1, apparatus 100 may include cohort database 116. A “cohort,” as used herein, is a group of individuals who share a common characteristic or experience. A “cohort database,” a used herein, is a data structure containing information about a plurality of individuals. In some embodiments, cohort database may include an EHR database of a hospital. A cohort may be a grouping of patients having relevant, identical, or similar user data 108 and/or abnormality datum to the user. A cohort may include retrospective patents examined previously over a predetermined period. “Retrospective users,” as used herein, are those part of a retrospective analysis or study. A retrospective analysis involves looking back at a group of patients who were previously treated or diagnosed to analyze outcomes, trends, or the effectiveness of treatments. These studies may include identifying patients who have already experienced a particular outcome or treatment and then tracing back in time to examine exposure to risk factors or the progression of their condition. Cohort database 116 may include a plurality of datasets, also referred to as tables herein, categorizing data such as user data of retrospective users, modalities retrospective users, clinical observations, enrichment, and the like. In an embodiment, a user data of retrospective users table may include a plurality of datasets, each indexing user data to retrospective users by time, demographics, symptoms, and the like.

Still referring to FIG. 1, a modalities table may include methods of treatment, or therapeutic approaches related to retrospective users. A “modality,” as used herein, is method or approach used for diagnosing, treating, or managing a health condition. Modalities include a wide array of techniques ranging from various diagnostic tests and medical imaging methods to different treatment and therapeutic interventions. Modalities may include various types of treatments such as surgical, pharmaceutical, behavioral interventions, and the like. Modalities may relate to radiology, cardiology, pathology, molecular omics (analysis of biological molecules such as DNA, RNA, proteins, metabolites, and the like), and the like. Modalities may include a time series of modalities and modality combinations. Modalities may include ECGs, Echocardiograms, CT scans, X-rays, and the like. A time series of modalities refers to the sequential use of various diagnostic or treatment methods over time to monitor and manage a patient's condition. For example, a patient with cardiac symptoms may undergo a resting ECG to determine a baseline of their heart's electrical activity. This initial modality may provide data for initial assessment and diagnosis. If the resting ECG suggests abnormal findings, the next step in the time series may include more extensive modalities such as an echocardiogram to visualize the heart's structure and function or a stress test ECG to assess how the heart performs under physical stress. Furthering the example, by incorporating modality combinations, a patient may have an ECG alongside an echocardiogram to correlate electrical and mechanical aspects of heart function or combine a stress test with imaging modalities to assess coronary artery disease. In some embodiments, data from time series of modalities and/or modality combinations may be analyzed by the processor 104 to detect patterns, correlations, predict outcomes, and tailor treatments using machine learning or other forms of artificial intelligence as described herein.

Still referring to FIG. 1, a clinical observation table may include data related to monitoring, recording, and interpretation of patients' clinical data over time in relation to one or modalities as described above. Clinical observations may include a detailed recording of patients' symptoms, the progression of their conditions, treatment responses, and any side effects or complications. Clinal observation data may include statistically significant clinical observations. Statistically significant clinical observations may refer to findings in clinical data that are unlikely to have occurred by chance and therefore suggest a real effect or association. A statistically significant observation may be indicated by a p-value. The p-value is a statistic that helps determine whether the results of a study are statistically significant. For example, a p-value of less than 0.05, may suggest a low probability that the observed results happened randomly. A statistically significant observation may be indicated by confidence intervals that may provide a range of values within which the true value is expected to fall a certain percentage of the time.

Still referring to FIG. 1, clinical observation data may include clinical significance data. Clinical significance data relates to the practical importance of a study's/modality findings in terms of their real-world impact on patient care, treatment outcomes, or decision-making processes in healthcare. Clinical significance data may include the magnitude of effect of a modality. Clinical significance data may include the patient outcome such as improvements in symptoms, quality of life, functional status, or other outcomes that patients perceive as beneficial or that lead to meaningful changes in their health status. Clinical significance data may include information related, cost-effectiveness, safety, side effects, generalizability (the applicability of results to various populations or settings can influence clinical significance), expert consensus, and the like. For example, recommendations from professional organizations or consensus among experts can influence perceptions of what is clinically significant.

Still referring to FIG. 1, clinical observation data may include link data. Link data may include correlations among statistically significant clinical observations, clinical significance data, user data 108 and the like. For example, link data may indicate a certain demographic of patients statistically experience a greater positive effect of a modality versus other demographics. Link data may be received through resources as described below, such as an AMC database. Link Data may be determined by processor 104 using machine learning techniques as described further below.

Still referring to FIG. 1, an enrichment table may include additional information and enhancements that are added to clinical data and/or user data of retrospective users to provide more context, depth, or value for analysis, decision-making, or research purposes within the healthcare field. Enrichment data may include medical annotations or labels indicating the presence of specific symptoms, diagnoses, medications, procedures, or outcomes. Enrichment data may include information that supplements user data of retrospective users with more detailed or specialized information. This may include laboratory test results, imaging studies, genetic data, patient-reported outcomes, and other relevant medical information. For example, enrichment data may add details about medication dosages, treatment protocols, adverse reactions, and comorbidities that may provide a more comprehensive picture of a patient's medical history and current health status. The enrichment table may categorize enrichment data based on user data of retrospective users, modalities, clinical observations and the like.

Still referring to FIG. 1, processor 104 may be configured to populate cohort database 116 using a web crawler to receive data or additional datum to index and categorize by tables as disclosed above. A “web crawler,” as used herein, is a program that systematically browses the internet for the purpose of Web indexing. The web crawler may be seeded with platform URLs, wherein the crawler may then visit the next related URL, retrieve the content, index the content, and/or measures the relevance of the content to the topic of interest. For example, processor 104 may generate a web crawler to scrape statistically significant clinical observations related to one or more modalities from a plurality of medical research websites. The web crawler may be seeded and/or trained with a reputable website to begin the search. A web crawler may be generated by processor 104. In some embodiments, the web crawler may be trained with information received from a third party through a user interface 112. For example, a health physician may seed the web crawler with websites and databases to search, and the type of data to extract, as an input through the user interface 112 as described above. In some embodiments, the web crawler may be configured to generate a web query. A web query may include a search criteria received from a third party. The search criteria may include an inclusion, exclusion, or combination thereof type of criteria. An inclusion criteria may include characteristics or conditions that must be applicable or present in query results. Examples of an inclusion criteria may include age range, specific medical diagnosis, certain laboratory values, and the like. An exclusion criteria may include characteristics or conditions that must be absent or non-applicable in query results. Examples of an exclusion criteria may include exclusion of certain comorbidities, use of specific medications, pregnancy or breastfeeding status, and the like.

Still referring to FIG. 1, processor 104 may implement an API (Application Programming Interface) to populate cohort database 116 by enabling an exchange and integration of datastores across various healthcare applications and systems across multiple geographical locations. API integration may allow for communication with a plurality of healthcare systems and databases for processor 104 aggregate data from in real time. In some embodiments, processor 104 may access academic medical center (AMC) databases that are specialized repositories that aggregate a wide range of clinical, educational, and research data associated with academic medical centers. For example, an AMC database may include data from clinical trials, biomedical research studies, genomic research, and other scientific investigations. In another example, an AMC database may include clinical information from patient care activities, including electronic health records (EHRs), laboratory results, imaging data, medication records, and more. This information may allow for the monitoring of treatment outcomes and facilitates quality improvement initiatives.

Still referring to FIG. 1, processor 104 may be configured to generate link data. A link machine-learning model may be configured to receive data for the cohort database 116 and classify certain elements, features, observations and the like to output link data. In some embodiments, link data may be generated by comparing vector embeddings of the user data to vector embeddings of the data in cohort database 116. For example, the link data may indicate that African American patients statistically show a better response to a certain heart medication compared to other demographics. In an embodiment, the machine learning model may receive user data of the retrospective users and implement a feature extraction algorithm to identify relevant features of interest such as specific details about the modalities (e.g., type and dosage of medication), patient demographics, and relevant clinical parameters. Processor 104 may use techniques like Recursive Feature Elimination (RFE) to identify and retain the most relevant features, eliminating noise in the data. In an example, user data of the retrospective users may include features like age, gender, race, blood pressure readings, cholesterol levels, medication dosage, treatment duration, concurrent conditions, lifestyle factors (e.g., smoking status, physical activity), and genetic markers. A feature extraction algorithm may include univariate analysis to evaluate the relationship between each independent feature and the treatment response. For example, a preliminary analysis may indicate that patients with higher baseline blood pressure levels are less likely to show improvement undergoing a specific modality. The machine-learning model may be configured to determine specific correlations focused on certain topics, such as demographics, effectiveness of a particular modality, and the like. The machine-learning model training data may include data correlating user data of the retrospective users to outcomes, such as ‘responded well’ or ‘did not respond well’ based on clinical criteria. A clinical criteria may include set of standards or guidelines used to make clinical decisions, derived from evidence-based research, expert consensus, or clinical practice guidelines. For example, a clinical criteria may include diagnostic criteria, treatment protocols, outcome measures, and other clinical indicators that help in assessing patient conditions, treatment efficacy, or health outcomes. Furthermore, various algorithms may be used for classification, such as logistic regression, decision trees, or more models like neural networks.

Still referring to FIG. 1, improvement to the link machine-learning model may be performed to enhance the accuracy of the generated outcome. For example, if the dataset, such as the user data 108, is small, techniques like SMOTE (Synthetic Minority Over-sampling Technique) may be used to generate synthetic data points, especially for underrepresented classes, to improve model training. In another embodiment, if the dataset is imbalanced (e.g., there are far more patients who respond to the treatment than those who do not), processor 104 may use techniques such as weighted classes to adjust the decision threshold to ensure the link machine-learning model does not become biased toward the majority class. The quantity of data that goes into generating the link data may vary and fluctuate based on a plurality of variables, such as the quantity of platforms visited by the WebCrawler, the implementation of feature extraction algorithms, and the like. Without the implementation of a machine-learning model, there would be a trade in the performance power of process, such as time and accuracy, in order to sort the data and generate link that are then used in a separate classification process, as described further below, in order to classify user data 108 to a cohort(s). The ability to continuously train a machine-learning model cable of learning to identify new trends or correlations within a fluctuating quantity of data is a benefit that would not be realized otherwise, without the tradeoff in performance efficiency.

Still referring to FIG. 1, in some embodiments, the link machine-learning model may include a classifier. A “classifier,” as used in this disclosure is a machine-learning model, such as a mathematical model, neural net, or program generated by a machine learning algorithm known as a “classification algorithm,” as described in further detail below, that sorts of inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Processor 104 and/or another device may generate a classifier using a classification algorithm, defined as a processes whereby a processor 104 derives a classifier from training data. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 1, processor 104 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)+P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Processor 104 may then calculate a likelihood table by calculating probabilities of different data entries and classification labels. Processor 104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 1, processor 104 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 1, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 7, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm:

$$l = \sqrt{{\sum }_{i = 0}^{n} {a}_{i}^{2}} ,$$

where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

Still referring to FIG. 1, cohort database 116 may include a preliminary cohort table. A preliminary cohort table may include cohorts of retrospective users received, using a process as described above, and categorized by various features. For example, processor 104 may receive, from a plurality of AMC databases, cohorts of patients based on a modality, symptom, and the like. Preliminary cohort table may also include cohorts generated by the processor 104 using methods as described further below as a functions of receiving and indexing user data of retrospective users. Preliminary cohort table may also include cohorts iteratively generated by the processor 104 from past applications of apparatus 100.

Still referring to FIG. 1, processor 104 is configured to generate a query input 120. A “query input,” as used herein, is data configured to specify what data should be fetched, updated, inserted, or deleted from a database. In some embodiments, query input 120 may be received from a user, such as a doctor or medical professional. In some embodiments, user may interact with user interface, such as a button, drop down, check box or the like, in order to “include” or “exclude” certain cohorts or sub cohorts from query input 120 or cohort of retrospective users. The query input 120 may include elements of user data 108 such as the symptoms, modalities, abnormality datum, or medical history of a user. The query input 120 may tell the cohort database 116 system what operation to perform and on what data. A query input 120 may include a query criteria 124. A “query criteria,” as used herein, is a is a condition or set of conditions specified in a database query that the data must meet to be selected or affected by the query. For example, a query input 120 may include instructions for the processor 104 to pull from the cohort database 116 user data of retrospective users 50 years old in age who suffered from diabetes and undergone weight loss medication as a result. A query criterion 124 may include inclusion, exclusion, or a combination thereof, as described above. Query input 120 may be received through the user interface 112 as described above.

Still referring to FIG. 1 a query input 120 may include a natural language database query. As used herein, a “natural language database query” is a data structure describing a request for patient data/user data, where the request is in a natural language form. As used herein, a “natural language form” is a combination and order of words, phrases, numbers, grammar, and syntax which may occur in human to human communication. As examples, a natural language form may be grammatically correct, may use slang, and may use abbreviations. A natural language form does not include computer code. A natural language database query may include, in non-limiting examples, a string of text input by a user, and/or an audio file including speech of a user. A natural language database query may include, in a non-limiting example, the statement “please generate a cohort of patients with Alzheimer's.” In another non-limiting example, a natural language database query may include the statement “please generate a cohort of patients at least 50 years old with b cell lymphoma.”

Still referring to FIG. 1, in some embodiments, apparatus 100 may receive natural language database query using a chatbot as described further below. For example, chatbot may interact with a third party, such as a health physician, by receiving inputs from a third party and outputting language to the third party. In some embodiments, chatbot may prompt a third party for a natural language database query. In some embodiments, chatbot may output text to a user. In some embodiments, chatbot may output audio to a user. In some embodiments, outputs of a chatbot may be determined using a language model. A language processing model may include a program automatically generated by computing device and/or language processing module to produce associations between one or more words extracted from at least a document and detect associations, including without limitation mathematical associations, between such words. Associations between language elements, where language elements include for purposes herein extracted words, relationships of such categories to other such term may include, without limitation, mathematical associations, including without limitation statistical correlations between any language element and any other language element and/or language elements. Statistical correlations and/or mathematical associations may include probabilistic formulas or relationships indicating, for instance, a likelihood that a given extracted word indicates a given category of semantic meaning. As a further example, statistical correlations and/or mathematical associations may include probabilistic formulas or relationships indicating a positive and/or negative association between at least an extracted word and/or a given semantic meaning; positive or negative indication may include an indication that a given document is or is not indicating a category semantic meaning. Whether a phrase, sentence, word, or other textual element in a document or corpus of documents constitutes a positive or negative indicator may be determined, in an embodiment, by mathematical associations between detected words, comparisons to phrases and/or words indicating positive and/or negative indicators that are stored in memory at computing device, or the like.

Still referring to FIG. 1, in some embodiments, the chatbot may include a large language model (LLM). LLM may include ChatGPT, GPT-2, GPT-3, GPT-4. LLM may include any suitable LLM. In some embodiments, LLM may be a global LLM. For example, LLM may be located on servers outside of a hospital's system. In some embodiments, LLM may be a local LLM. In some embodiments, use of an LLM running on a local computing device such as processor 104 may improve security of apparatus 100. For example, use of an LLM running on processor 104 and/or another local device may make it unnecessary to send sensitive data over the internet, reducing the risk of unauthorized access to such data. In another example, use of an LLM running on processor 104 and/or another local device may improve the ease with which computational resources may be allocated to an LLM and/or allow for ease of fine-tuning and/or higher security in a fine-tuning process. For example, use of a local LLM may make it unnecessary for sensitive data in a dataset used for fine-tuning to be sent over the internet, which would pose a security risk. LLM may be located on servers within a hospital system or other external platforms. In some embodiments, use of a remote LLM may allow for higher scalability than a local LLM. In some embodiments, parameters of LLM may be chosen such that LLM may be run on a local system. For example, the expected input/output may be set to English Language. Additionally, single GPU training may be used.

Still Referring to FIG. 1, processor 104 is configured to generate a cohort of retrospective users 128 as a function of the query input 120. Generating a cohort of retrospective users 128 may include compiling a list of relevant patients highlighting key elements of user data 108 and the like associated with each patient that correlates to the user. For example, a query input 120 may include a modality a physician would like the user to undergo. Processor 104 may compile retrospective users with similar medical histories to the user and highlight the success rate of the modality, statistically significant side effects, and the like. The cohort of retrospective users 128 may be displayed through the user interface 112. In some embodiments, a cohort of retrospective users 128 may be federated. A federated cohort refers to an inclusive group of study participants across various populations. A federated cohort may include patients from a wide range of ethnic backgrounds, age groups, socioeconomic statuses, genders, and other demographic variables to ensure that the results are generalizable and applicable to a broad population, not biased towards a specific group. In another embodiment, a cohort of retrospective users 128 may include a premium cohort. A premium cohort may include a select group of patients receiving treatment at specific, highly regarded hospital and are under the care of top-rated, yet anonymized, physicians associated with Academic Medical Centers (AMCs). A premium cohort may indicate that the data collected is of high quality, given the advanced care environment. Research derived from premium cohorts may provide valuable insights into the effectiveness of treatments, patient outcomes, and healthcare practices at top-tier medical institutions.

Still referring to FIG. 1, generating a cohort may include classifying user data 108 to one or more cohorts of the preliminary cohort table as described above. For example, processer may implement a machine-learning model such as a preliminary classifier to receive user data 108 as an input and output cohort matched to the user. The training data may include a plurality of user data correlated to a plurality of preliminary cohorts (cohorts previously generated and stored in preliminary data of cohort database 116). In another embodiment, processor 104 may use a fuzzy set inference system to match user data 108 to one or mor preliminary cohorts or cohorts as generated by the processor 104 as described further below. For example, processor 104 may identify and select key medical attributes from retrospective users' histories, such as symptoms severity or responses to treatments. For each attribute, processor 104 may then apply fuzzy logic to assign a degree of membership, transforming data into a fuzzy numerical scale that reflects the nuances of medical conditions. Following this, processor 104 may aggregate these fuzzy values for each retrospective patient to construct a comprehensive fuzzy profile, encapsulating the multifaceted nature of their medical history. Concurrently, processor 104 nay perform a similar aggregation for existing patient cohorts, creating fuzzy set representations for these groups based on the collective data of their members. The processor 104 may calculate similarity indices between the fuzzy profile of the current patient and those of the retrospective cohorts. By assessing the degree of overlap or closeness between these fuzzy sets, the processor 104 may identify which cohort(s) most closely align with the user's medical history.

Still referring to FIG. 1, in some embodiments, processor 104 may generate a vector embedding 132 to use as an AI generated query criteria 124 to generate a cohort of retrospective users 128 based on a query input 120. Given any individual modality or combinations of modalities from the query input, processor 104 may implement supervised, unsupervised or self supervised neural networks (NNs) or generative artificial intelligence technology to build a vector embedding 132 or other statistical representation of an individual modality like an ECG or a CT or MRI or XRay or whole slide image or a gene panel or Illumina output or patient note or time series of structured data (ICD CPT Drug codes). Additionally a vector embedding 132 may be built for a combination of one or more of these modalities linked at the patient level using multimodal neural networks as described further below. Vector embedding 132 or other representations may allow processor 104 to define neighborhoods of embeddings or representation instances based on cosine, Euclidean, Mahalanobis distances, combinations thereof, and the like. In various embodiments, neighborhoods may be calculated using embeddings or features. For example, in some embodiments, neighborhoods may be calculated based on distance metrics using features. For example, in some embodiments, neighborhoods may be calculated based on distance metrics using embeddings. A person of ordinary skill in the art, after having reviewed the entirety of this disclosure, would appreciate that the methods for determining cohorts based on vector embeddings described throughout this disclosure could be analogously applied to determining cohorts based on features extracted from user data 108. To define neighborhoods, a threshold value may be set for the distance metrics. If the distance between two embeddings or features is below or above this threshold, they may be considered part of the same neighborhood. For example, a threshold of 0.5 may be set for cosine similarity, two patients whose data embeddings or features have a cosine similarity greater than 0.5 with each other may be considered part of the same neighborhood. A threshold may be predefined or dynamically determined based on the data distribution. Retrospective users of the same neighborhood may be aggregated to form a cohort. Aggregation may be statistical, summarizing the features for each cohort using mean, median, standard deviation, or other relevant metrics that provide insight into the commonalities within the cohort. For example, in the context of ECG data, specific ECG features like heart rate, QRS duration, QT interval, or other characteristic waveforms that the embeddings or features captured may be aggregated. In another embodiment, aggregation may include identifying patterns that are prevalent within a cohort. For example, if a cohort is characterized by a specific pattern in the ECG waveform that suggests a certain cardiac condition, this pattern may become a defining characteristic of the cohort. In some embodiments, aggregated data and identified patterns may then correlated with clinical interpretations. For example, if the aggregated ECG features of a cohort align with known markers of a specific cardiac condition, this association may help to clinically characterize the cohort. Each formed cohort may then be characterized based on common features or patterns shared among its members. For example, if a cohort is formed based on vector embeddings or features derived from ECG data, the cohort may represent a group of patients with similar cardiac profiles.

With continued reference to FIG. 1, vector embedding 132 are a type of representation that converts items, such as words, images, or any object, into a vector of numbers. This representation captures the essential features of the items in a continuous vector space, where the geometric relationships between the vectors reflect the similarities or relationships between the items. Such vector and/or embedding may include and/or represent an element of a vector space; a vector may alternatively or additionally be represented as an element of a vector space, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. A vector may be represented as an n-tuple of values, where n is one or more values, as described in further detail below; a vector may alternatively or additionally be represented as an element of a vector space, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 7, 15] may be treated as equivalent, for purposes of this disclosure, as a vector represented as [1, 2, 3]. Vectors may be more similar where their directions are more similar, and more different where their directions are more divergent, for instance as measured using cosine similarity as computed using a dot product of two vectors; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute 1 as derived using a Pythagorean norm:

$$l = \sqrt{{\sum }_{i = 0}^{n} {{a}_{i}}^{2}} ,$$

where ai is attribute number i of the vector. Scaling and/or normalization may function to make vector comparison independent of absolute quantities of attributes, while preserving any dependency on similarity of attributes. A two-dimensional subspace of a vector space may be defined by any two orthogonal vectors contained within the vector space. Two-dimensional subspace of a vector space may be defined by any two orthogonal and/or linearly independent vectors contained within the vector space; similarly, an n-dimensional space may be defined by n vectors that are linearly independent and/or orthogonal contained within a vector space. A vector's “norm’ is a scalar value, denoted ∥α∥ indicating the vector's length or size, and may be defined, as a non-limiting example, according to a Euclidean norm for an n-dimensional vector α as:

$$‖ a ‖ = \sqrt{{\sum }_{i = 0}^{n} {a}_{i}^{2}}$$

In an embodiment, and with continued reference to FIG. 1, each user data 108 element or query input 120 may be represented by a dimension of a vector space; as a non-limiting example, each element of a vector may include a number representing an enumeration of co-occurrences of first user data 108 element or query input 120 represented by the vector with second user data 108 element or query input 120. Alternatively, or additionally, dimensions of vector space may not represent distinct user data 108 elements, in which case elements of a vector representing a first user data 108 element or query input 120 may have numerical values that together represent a geometrical relationship to a vector representing a second user data 108 element or query input 120, wherein the geometrical relationship represents and/or approximates a semantic relationship between the first user data 108 element or query input 120 and the second user data 108 element or query input 120. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below.

Any vectors as described herein may be scaled, such that each vector represents each attribute along an equivalent scale of values. In an embodiment associating user data 108 to one another as described above may include computing a degree of vector similarity between a vector representing each user data 108 element or query input 120 and a vector representing another user data 108 element or query input 120; vector similarity may be measured according to any norm for proximity and/or similarity of two vectors, including without limitation cosine similarity. As used in this disclosure “cosine similarity” is a measure of similarity between two-non-zero vectors of a vector space, wherein determining the similarity includes determining the cosine of the angle between the two vectors. Cosine similarity may be computed as a function of using a dot product of the two vectors divided by the lengths of the two vectors, or the dot product of two normalized vectors. For instance, and without limitation, a cosine of 0° is 1, wherein it is less than 1 for any angle in the interval (0, π) radians. Cosine similarity may be a judgment of orientation and not magnitude, wherein two vectors with the same orientation have a cosine similarity of 1, two vectors oriented at 60° relative to each other have a similarity of 0, and two vectors diametrically opposed have a similarity of −1, independent of their magnitude. As a non-limiting example, vectors may be considered similar if parallel to one another. As a further non-limiting example, vectors may be considered dissimilar if orthogonal to one another. As a further non-limiting example, vectors may be considered uncorrelated if opposite to one another. Additionally, or alternatively, degree of similarity may include any other geometric measure of distance between vectors.

Still referring to FIG. 1, in an example, incorporating vector embedding 132, a modality query input 120 may be utilized to extract relevant user data 108 from retrospective users from the cohort database 116, focusing on those who underwent a particular treatment or diagnostic procedure related to the modality query input 120. In some embodiments, relevant user data 108 from cohort database 116 may be referred to as cohort data. Relevant user data 108 from cohort database 116 may be converted into vector embeddings (e.g., set of vector embeddings) using any method for determining vector embeddings that is disclosed in this disclosure. This data, encompassing both textual reports and imaging, may then be transformed into a high-dimensional vector space. For textual data, a natural language processing (NLP) technique like Word2Vec may be employed to convert, for example, MRI reports into meaningful vector embedding 132, capturing the semantic essence of each report. Concurrently, MRI images may be processed through a convolutional neural network to distill key image features into compact vector representations. These embeddings, representing both textual and visual data, may then be analyzed using clustering algorithms to group patients into distinct cohorts based on the similarity of their embeddings, effectively identifying clusters that correspond to the queried modality. Once the vector embedding 132 are derived from retrospective patient data in response to a specific modality query, processor 104 may implement a classifier to categorize these embeddings based on the associated outcomes, for example, negative, neutral, or positive, related to the modality. The classifier may be trained to detect patterns in the embeddings that correspond to each outcome category and sort the patient vectors into distinct groups/cohorts. For example, embeddings that include data from patients with positive responses to the treatment modality may be grouped together, the same may occur for patients with neutral and negative responses.

Still referring to FIG. 1, a single modality or combination of modalities can be represented through multiple AI/machine learning derived embeddings or representations as described above. Different AI algorithms may analyze the same dataset and produce varied embeddings based on their unique designs, intents, and methodologies. These diverse approaches may highlight and prioritize different facets of the modality or modality combination within the embeddings they create. This allows for a variety of options that physicians may choose from when defining inclusion/exclusion criteria for a cohort. This flexibility may allow healthcare providers to select the AI approach that most closely aligns with their clinical objectives, enhancing the precision and relevance of the cohort matching process for current patients.

With continued reference to FIG. 1, cohort generation and hypothesis verification may be implemented as described in U.S. patent application Ser. No. 18/648,059 (having attorney docket number 1518-129USU1), filed on Apr. 26, 2024, and titled “APPARATUS AND METHODS FOR GENERATING DIAGNOSTIC HYPOTHESES BASED ON BIOMEDICAL SIGNAL DATA,” the entirety of which is incorporated herein by reference.

With continued reference to FIG. 1, in some embodiments, processor 104 may extract one or more features from user data 108. As used herein, “feature extraction” refers to a process of identifying non-domain specific features within an initial data set and isolating those features for subsequent processing. In some embodiments, processor 104 may extract one or more features from user data 108 using a feature extraction model. Feature extraction model may include a machine-learning model. In some embodiments, feature extraction model may be trained using unsupervised learning. As a non-limiting example, feature extraction model may be trained with feature extraction training data, wherein the feature extraction training data may include unlabeled sets of user data 108 (such as ECGs, CT scans, MRIs, X-rays, EHR data, or the like). In some embodiments, feature extraction model may be trained using supervised learning. In one or more embodiments, feature extraction model may be trained with feature extraction training data including user data 108 correlated to features. In some embodiments, feature extraction model may comprise a plurality of feature extraction models. In some embodiments, each of the plurality of feature extraction models may be trained specifically for particular modalities of user data 108. For example, one feature extraction model may be trained to extract features from ECGs while another may be trained to extract features from EHR data, or any of the other modalities mentioned in this application. In some embodiments, training data may be specialized for each of these models; for example, an EHR feature extraction model may be trained using training data comprising EHR data correlated to features or training data comprising unlabeled EHR data. In some embodiments, feature extraction model may use one or more clustering algorithms. For example, clustering algorithms may include affinity propagation, agglomerative clustering, BIRCH, DBSCAN, K-means clustering, mini-batch k-means, mean shift, OPTICS, spectral clustering, mixture of Gaussians, or the like. In some embodiments, feature extraction model may use any clustering and/or classification algorithm disclosed in this disclosure. In some embodiments, feature extraction and/or vector embedding generation may be consistent with feature extraction and/or vector embedding generation disclosed in U.S. Non-provisional application Ser. No. 18/230,477 (Attorney Docket No. 1517-051USU1), filed on Aug. 4, 2023, and entitled “APPARATUS AND METHODS FOR EXPANDING CLINICAL COHORTS FOR IMPROVED EFFICACY OF SUPERVISED LEARNING,” the entity of which is incorporated herein by reference.

With continued reference to FIG. 1, in some embodiments, vector embeddings may be determined for any features extracted from user data 108 by feature extraction model.

Still referring to FIG. 1, processor 104 may be configured to extract and quantify specific anatomical, structural, or molecular features and biomarkers within a person's body, such as the number of nuclei in a particular region or various omics data (genomics, proteomics, and the like). These measurements may be used as parameter(s) in a query criteria 124 to generate a cohort using any method as described herein. Processor 104 may extract these features and biomarkers from user data 108 using a language processing method as described above or an optical character recognition method as described further below. In this embodiment, processor 104 may be configured to identify and calculate distinct internal characteristics or markers, which may range from cellular details to broader molecular profiles, providing a detailed snapshot of the patient's internal state. By integrating these specific, quantifiable factors into query criteria 124, healthcare professionals can refine their search or analysis parameters, enabling more targeted and nuanced investigations into patient data. Machine-learning models may be configured to recognize patterns in the quantified data and classify retrospective users based on the presence, absence, or quantity of certain biomarkers. For example, patients may be grouped according to specific molecular signatures or anatomical features identified through the analysis.

Still referring to FIG. 1, in extracting and quantifying features and biomarkers, processor 104 may implement a machine-learning model to conduct a temporal analysis on time-series data. For example, time-series data may include cardiac CT scans, showing the heart's movement throughout the cardiac cycle. Machine-learning models, including recurrent neural networks (RNNs) or convolutional LSTM (Long Short-Term Memory) networks, may be configured to analyze the time-series data to assess cardiac function, such as ejection fraction or wall motion. Functional measurements such as ejection fraction may be computed to enable the detection and grading of functional heart disorders. In an example, processor 104 may collect a series of cardiac CT scans that capture the heart's movement throughout the cardiac cycle. This may include receiving multiple scans at different times during the heart's contraction and relaxation phases to create a complete cycle. Processor 104 may then configure a machine-learning model to process sequences of images. For example, for a convolutional LSTM, processor 104 may define convolutional layers to handle the spatial data within each image, followed by LSTM layers to manage temporal dependencies across the sequence. Training data may include data such as measured ejection fractions or documented wall motion abnormalities correlated to time series data to teach the model how to accurately predict these measurements from the CT scan sequences. The model may identify and quantify changes in the heart's structure and motion throughout the cycle. The model may automatically calculate functional measurements, such as the ejection fraction. The model may apply the extracted measurements to diagnose and grade cardiac disorders. For example, a reduced ejection fraction identified by the model may indicate heart failure. The model may be continuality trained on user data to refine the its accuracy and sensitivity.

Still referring to FIG. 1, in extracting and quantifying features and biomarkers, processor 104 may compile a wide range of user data 108 including imaging data such as MRIs, CT scans, and the like. Processor 104 may use a machine-learning model, such as convolutional neural networks (CNNs), to tract and quantify biomarkers. For example, CNNs may be trained to analyze the image data to identify and quantify features indicative of heart diseases, such as the presence of coronary artery calcification, the thickness of the heart walls, or the size of the heart chambers. CNNs may be configured to recognize patterns and anomalies in the cardiac CT images. For example, a CNN may be trained to detect calcified plaques in the coronary arteries, which are a marker for coronary artery disease. The CNN may then quantify the extent of calcification, providing a score that correlates with disease severity. Additionally, the CNN may output quantitative metrics such as calcification scores, chamber volumes, or wall thickness measurements which may be used in a query criteria 124. For example, processor 104 may be configured to display the metrics through the user interface 112, prior to generating a cohort or as an iterative step for refining cohort results. In an example a query input 120 may include “elect patients with an ejection fraction below 40% and significant coronary calcification.”

Still referring to FIG. 1, in extracting and quantifying features and biomarkers, processor 104 may be configured to implement machine-learning to create measurements of anatomical or other biomarkers relevant to one or more organs of a patient. Machine-learning models may be used to integrate data from multiple modalities, enhancing the processor's 104 capacity to generate a detailed understanding of a patient's health. For example, a machine-learning model may analyze cardiac CT images to assess coronary calcification and chamber size, while simultaneously integrating data from echocardiograms, MRI, or PET scans to provide additional insights into the heart's structure, function, and metabolic state. While the CT provides detailed structural information, MRI offers insights into tissue characterization, and echocardiograms contribute dynamic functional data. Analyzing these modalities together, the machine-learning model and/or processor 104 may identify and quantify a broader range of biomarkers, for example, integrating plaque characterization from CT, myocardial scarring from MRI, and ventricular function from echocardiography. This method may be applied to related structures or organs. For example, processor 104 may use a machine-learning model to assess the aorta for signs of aneurysm or dissection in the context of overall cardiovascular health. This data may be used to update query results by narrowing down the query criteria 124 as described above. For example, query input 120 may include “identify patients with left ventricular hypertrophy, reduced ejection fraction, and evidence of myocardial fibrosis.”

Still referring to FIG. 1, in an embodiment, processor 104 may use a machine-learning model to generate a biomarker criteria to be additionally implemented as categorical variables in a query criteria 124/query of cohort database 116. For example, in relation to cardiac health, machine-learning may be used to classify and categorize various conditions or features detected across different modalities, such as identifying types of myocardial tissue (healthy, ischemic, necrotic) in MRI scans or classifying the severity of coronary artery stenosis in CT images. These classifications may be encoded as categorical variables, representing distinct, clinically relevant categories that can be utilized alongside other parameters, such as biomarker measurements in querying the cohort database 116. In an example, a machine-learning model may analyze echocardiogram videos to classify ventricular function as normal, mildly impaired, or severely impaired, while concurrently assessing cardiac CT scans to categorize the extent of coronary calcification. When combined with a biomarker measurement such as ejection fraction or plaque volume, these categorical classifications may provide a multidimensional dataset that can be queried. An example of query input 120, may include “select patients with severe coronary calcification, mildly impaired ventricular function, and evidence of myocardial scarring.”

Still referring to FIG. 1, in extracting data form user data 108 in the process of generating biomarker measurements as described above, processor 104 may implement an optical character recognition or optical character reader (OCR) includes automatic conversion of images of written (e.g., typed, handwritten or printed text) into ma that chine-encoded text. In some cases, recognition of at least a keyword from an image component may include one or more processes, including without limitation optical character recognition (OCR), optical word recognition, intelligent character recognition, intelligent word recognition, and the like. In some cases, OCR may recognize written text, one glyph or character at a time. In some cases, optical word recognition may recognize written text, one word at a time, for example, for languages that use a space as a word divider. In some cases, intelligent character recognition (ICR) may recognize written text one glyph or character at a time, for instance by employing machine learning processes. In some cases, intelligent word recognition (IWR) may recognize written text, one word at a time, for instance by employing machine learning processes.

Still referring to FIG. 1, in some cases OCR may be an “offline” process, which analyses a static document or image frame. In some cases, handwriting movement analysis can be used as input to handwriting recognition. For example, instead of merely using shapes of glyphs and words, this technique may capture motions, such as the order in which segments are drawn, the direction, and the pattern of putting the pen down and lifting it. This additional information can make handwriting recognition more accurate. In some cases, this technology may be referred to as “online” character recognition, dynamic character recognition, real-time character recognition, and intelligent character recognition.

Still referring to FIG. 1, in some cases, OCR processes may employ pre-processing of image component. Pre-processing process may include without limitation de-skew, de-speckle, binarization, line removal, layout analysis or “zoning,” line and word detection, script recognition, character isolation or “segmentation,” and normalization. In some cases, a de-skew process may include applying a transform (e.g., homography or affine transform) to image component to align text. In some cases, a de-speckle process may include removing positive and negative spots and/or smoothing edges. In some cases, a binarization process may include converting an image from color or greyscale to black-and-white (i.e., a binary image). Binarization may be performed as a simple way of separating text (or any other desired image component) from a background of image component. In some cases, binarization may be required for example if an employed OCR algorithm only works on binary images. In some cases. a line removal process may include removal of non-glyph or non-character imagery (e.g., boxes and lines). In some cases, a layout analysis or “zoning” process may identify columns, paragraphs, captions, and the like as distinct blocks. In some cases, a line and word detection process may establish a baseline for word and character shapes and separate words, if necessary. In some cases, a script recognition process may, for example in multilingual documents, identify script allowing an appropriate OCR algorithm to be selected. In some cases, a character isolation or “segmentation” process may separate signal characters, for example character-based OCR algorithms. In some cases, a normalization process may normalize aspect ratio and/or scale of image component.

Still referring to FIG. 1, in some embodiments an OCR process will include an OCR algorithm. Exemplary OCR algorithms include matrix matching process and/or feature extraction processes. Matrix matching may involve comparing an image to a stored glyph on a pixel-by-pixel basis. In some case, matrix matching may also be known as “pattern matching,” “pattern recognition,” and/or “image correlation.” Matrix matching may rely on an input glyph being correctly isolated from the rest of the image component. Matrix matching may also rely on a stored glyph being in a similar font and at a same scale as input glyph. Matrix matching may work best with typewritten text.

Still referring to FIG. 1, in some embodiments, an OCR process may include a feature extraction process. In some cases, feature extraction may decompose a glyph into features. Exemplary non-limiting features may include corners, edges, lines, closed loops, line direction, line intersections, and the like. In some cases, feature extraction may reduce dimensionality of representation and may make the recognition process computationally more efficient. In some cases, extracted feature can be compared with an abstract vector-like representation of a character, which might reduce to one or more glyph prototypes. General techniques of feature detection in computer vision are applicable to this type of OCR. In some embodiments, machine-learning process like nearest neighbor classifiers (e.g., k-nearest neighbors algorithm) can be used to compare image features with stored glyph features and choose a nearest match. OCR may employ any machine-learning process described in this disclosure. Exemplary non-limiting OCR software includes Cuneiform and Tesseract. Cuneiform is a multi-language, open-source optical character recognition system originally developed by Cognitive Technologies of Moscow, Russia. Tesseract is free OCR software originally developed by Hewlett-Packard of Palo Alto, California, United States.

Still referring to FIG. 1, in some cases, OCR may employ a two-pass approach to character recognition. Second pass may include adaptive recognition and use letter shapes recognized with high confidence on a first pass to recognize better remaining letters on the second pass. In some cases, two-pass approach may be advantageous for unusual fonts or low-quality image components where visual verbal content may be distorted. Another exemplary OCR software tool include OCRopus. OCRopus development is led by German Research Centre for Artificial Intelligence in Kaiserslautern, Germany. In some cases, OCR software may employ neural networks, for example neural networks.

Still referring to FIG. 1, in some cases, OCR may include post-processing. For example, OCR accuracy can be increased, in some cases, if output is constrained by a lexicon. A lexicon may include a list or set of words that are allowed to occur in a document. In some cases, a lexicon may include, for instance, all the words in the English language, or a more technical lexicon for a specific field. In some cases, an output stream may be a plain text stream or file of characters. In some cases, an OCR process may preserve an original layout of visual verbal content. In some cases, near-neighbor analysis can make use of co-occurrence frequencies to correct errors, by noting that certain words are often seen together. For example, “Washington, D.C.” is generally far more common in English than “Washington DOC.” In some cases, an OCR process may make us of a priori knowledge of grammar for a language being recognized. For example, grammar rules may be used to help determine if a word is likely to be a verb or a noun. Distance conceptualization may be employed for recognition and classification. For example, a Levenshtein distance algorithm may be used in OCR post-processing to further optimize results.

Still referring to FIG. 1, in some embodiments, a cohort may be generated or modified based on an intersection or union of one or more pre-existing or dynamically created cohorts. For example, a cohort may be generated based on the intersection or union of one or preliminary cohorts, as described above, or cohorts generated based on the query input. In response to query input 120, processor 104 may retrieve analogous preliminary cohorts from the cohort database. For example, query input 120 may instruct the inclusion of patients with brain tumor MRI scans and exclusion of patients who received chemotherapy within the past year. Processor 104 may identify cohort with brain tumor MRI scans and another cohort with records of chemotherapy treatments. Processor 104 may then intersect these cohorts to find patients common in both groups. With the exclusion criteria applied, the processor 104 forms a new cohort 128, dynamically generated based on the query input. The new cohort 128 is now a subset of the original MRI scan cohort, refined by the exclusion criteria. The new cohort 128 may be analyzed to extract insights, study patterns, or evaluate treatment outcomes. For example, a health provider may investigate the efficacy of non-chemotherapy treatments among the identified patients.

Still referring to FIG. 1, in some embodiments, processor 104 may additionally generate vector embeddings/statistical representations, referred to as patient embedding herein, of a user based on the time series of modalities and modality combinations for application in a query criteria 124 as described above. A patient embedding may include holistic representations that encapsulate the entire spectrum of a patient's interactions with various medical modalities over time. Processor 104 may implement machine learning algorithms to integrate features from user data 108 including single modalities, combinations of modalities, and the progression of these modalities over time, to generate a singular, multidimensional representation of a user's medical history. For example, a patient embedding may combine information from their cardiac CT scans, MRI results, echocardiography data, and lab tests, reflecting not just the state of their cardiac health but providing insights into their overall health status. Generating the patient embedding may use a vector space analysis process as described above. Vector embedding methods include those as disclosed in U.S. patent application Ser. No. 18/230,043 (having attorney docket number 1518-102USU1), filed on Aug. 3, 2023, and titled “APPARATUS AND A METHOD FOR GENERATING A DIAGNOSTIC LABEL,” the entirety of which is hereby incorporated by reference.

Still referring to FIG. 1, processor 104 may be configured to rank and statistically score different diagnoses based on enrichments-based AI and non-AI based neighborhoods and cohorts. In the context of comorbidity or any multifaceted medical dataset, ranking diagnoses may include evaluating and prioritizing different medical conditions based on certain criteria. These criteria may be their prevalence, severity, or the urgency of intervention needed within a patient population. Such a criteria may be received through a query input 120 or from an AMC database, and the like. For example, processor 104 may define patient cohorts based on various criteria, using both AI-based clustering techniques, such as K-means or hierarchical clustering on patient embeddings, and non-AI methods based on demographic or clinical criteria. Processor 104 may implement a feature extraction process as described above to extract relevant features from patient data, including diagnoses, comorbidities, treatment histories, outcomes, and potentially demographic and lifestyle factors. Processor 104 may then identify which features are most predictive of patient outcomes or treatment responses using techniques like feature importance from tree-based models or L1 regularization. Tree-based models may include decision trees, random forests, and gradient boosting machines, and the like may perform feature selection by prioritizing the most informative features at the top nodes of the trees. For instance, in a random forest, the importance of each feature can be evaluated based on how much it contributes to the reduction of variance or impurity in the nodes. In the context of comorbidities, tree-based models may identify and rank the most significant conditions affecting patient outcomes by learning complex patterns and interactions between various diagnoses. “L1 regularization,” commonly associated with Lasso regression, is a technique that introduces a penalty equivalent to the absolute value of the magnitude of coefficients. This encourages a machine-learning model to not only fit the data but also to keep the model coefficients (feature weights) as small as possible. By applying L1 regularization, the machine-learning model may differentiate between primary and secondary or less significant diagnoses, providing a ranked importance of various conditions, which can be crucial for managing patients with multiple comorbidities. A machine-learning model for ranking and scoring may implement tree-based models or L1 regularization. In some embodiments, the machine-learning model may implement both tree-based methods for their robustness and interpretability and L1 regularization for its sparse feature selection capability. For example, a gradient boosting model with built-in L1 regularization could be employed. The machine-learning model may be trained on historical patient data received from the cohort database, learning to predict outcomes or severity based on a comprehensive set of patient features, including a range of diagnoses. The machine-learning model may then score and rank patient diagnoses, providing a nuanced view of each patient's medical profile. The score or ranking for each diagnosis for a given patient may indicate the diagnosis's severity, prevalence, or urgency. This ranking may inform clinical decision-making, highlighting which conditions should be prioritized for treatment or further investigation. These rankings and scores may be displayed through the user interface 112 as described above.

Still referring to FIG. 1, apparatus 100 may allow for third parties, physicians, to ask, through query input 120, cohorts built from neighborhoods of multiple modalities taken together i.e. neighborhoods built from combined modalities. For example, considering a user's ECG (applicable analogously to any other patient modality or modality combination), for each comorbidity (D) there is a specific list of cohorts “C_{1D},C_{2D},C_{3D}” that is either searchable within the cohort database 116 or that the physician has the processor 104 create creates such that C_{1D},C_{2D},C_{3D}, are all clinically relevant groups of patients i.e. clinically relevant to the diagnosis D being considered The physician perform a parallel query for enrichments from clinically relevant cohorts for many indications/comorbidities/diagnoses all at once; i.e. not just one indication/comorbidity/diagnosis. The physician may also input AI as well as non-AI neighborhoods within each such clinically relevant cohort, for each such indication/comorbidity/diagnosis they have in mind. The physician may also perform differential diagnoses then by studying all the enrichments.

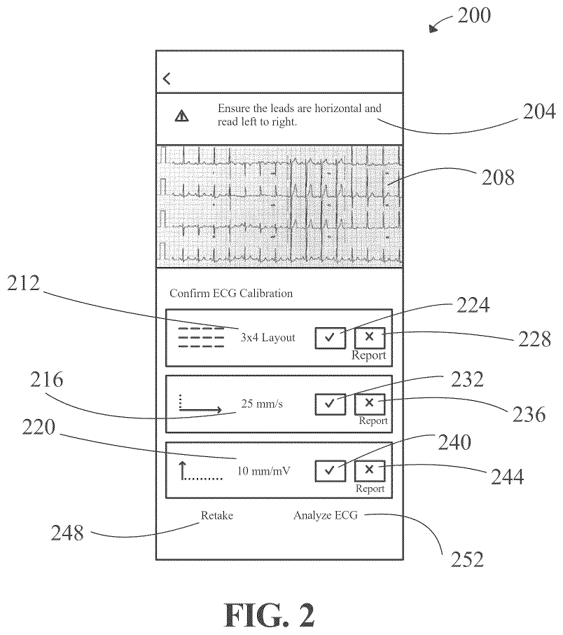

Referring now to FIG. 2, an exemplary embodiment of a user interface 200 is provided. User interface 200 may be configured to receive user data including ECG data. User interface 200 may include prompt 204. Prompt 204 may include, for example, instructions for a user/third party. For example, instructions may describe to user/third party how to properly input calibration datum. As used herein, a “calibration datum” is a category of a signal, a parameter of a signal, an orientation of a signal, a scale of a signal, or a combination thereof. Such a category of a signal may include, in a non-limiting example, an ECG (as opposed to another type of signal). Such a parameter of a signal may include, in a non-limiting example, a number of leads used to generate ECG data. Such an orientation of a signal may include, in a non-limiting example, an ECG being horizontal, and reading left to right. Such a scale of a signal may include, in a non-limiting example, a number of mm/s of a physical record of an ECG. User interface 200 may include image 208. Image 208 may include an image of a signal as described above. Image 208 may include a raw image and/or an image which has undergone one or more processing steps as described above. User interface 200 may include one or more elements of calibration data such as elements 212, 216 and 220. User interface 200 may include one or more interactive elements used to determine a calibration datum such as elements 224, 228, 232, 236, 240, and 244. User interface 200 may include an interactive element which may be used to cause apparatus 100 to capture a new image, such as element 248. User interface 200 may include an interactive element which may be used to initiate one or more steps described herein, such as element 252.

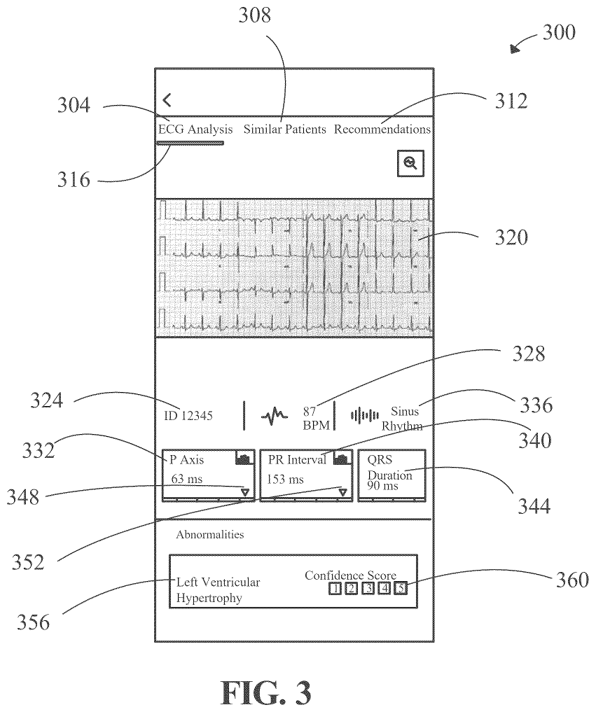

Referring now to FIG. 3, an exemplary embodiment of a user interface 300 is provided. User interface 300 may include one or more interactive elements by which a user may select varying functions of apparatus 100, such as elements 304, 308, and 312. User interface 300 may include an element which indicates a function currently selected, such as element 316. User interface 300 may include image 320. Image 320 may include an image of a signal as described above. Image 320 may include a raw image and/or an image which has undergone one or more processing steps as described above. User interface 300 may include an identification number 324 for a particular signal and/or image. User interface 300 may include one or more signal metrics, such as signal metrics 328, 332, 336, 340, and 344. User interface 300 may include one or more signal metric positions, such as signal metric positions 348 and 352. User interface 300 may include one or more elements of abnormality data such as abnormality datum 356 and/or one or more abnormality datum confidence scores such as abnormality datum confidence score 360 associated with such abnormality data. Singal metric positions and abnormality data may be used to classify a user to a cohort using methods as described in this disclosure.

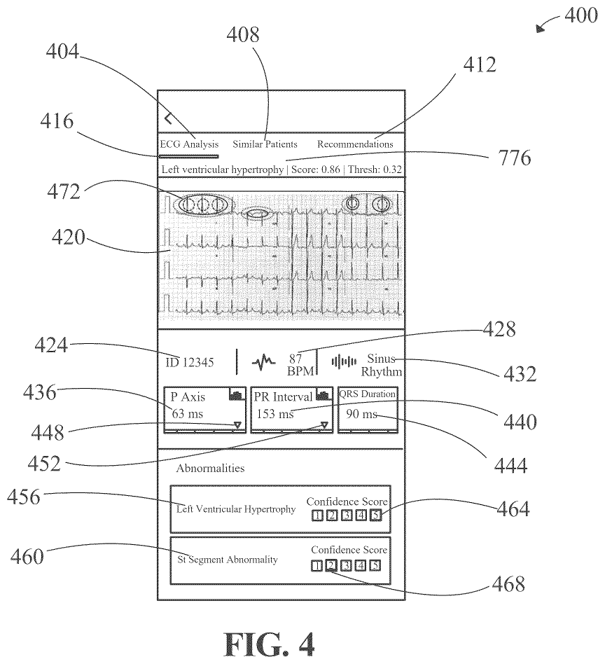

Referring now to FIG. 4, an exemplary embodiment of a user interface 400 is provided. User interface 400 may include one or more interactive elements by which a user may select varying functions of apparatus 100, such as elements 404, 408, and 412. User interface 400 may include an element which indicates a function currently selected, such as element 416. User interface 400 may include image 420. Image 420 may include an image of a signal. A “signal” is a physical record of medical data of a subject. In some embodiments, a signal may include a measurement of activity of a subject's heart. In some embodiments, a signal may include ECG data. In some embodiments, a signal may include time series data. In some embodiments, a signal may include a plurality of parallel recordings of time-series data, such as in a 12 lead ECG.

Image 420 may include a raw image and/or an image which has undergone one or more processing steps as described above. User interface 400 may include an identification number 424 for a particular signal and/or image. User interface 400 may include one or more signal metrics, such as signal metrics 428, 432, 436, 440, and 444. User interface 400 may include one or more signal metric positions, such as signal metric positions 448 and 452. User interface 400 may include one or more elements of abnormality data such as abnormality data 456 and 460 and/or one or more abnormality datum confidence scores such as abnormality datum confidence scores 464 and 468 associated with such abnormality data. User interface 400 may include map 442 indicating regions which contribute to determination of an abnormality datum. User interface 400 may include an indicator 446 indicating which abnormality datum map 442 is referring to and/or an associated abnormality datum confidence score.

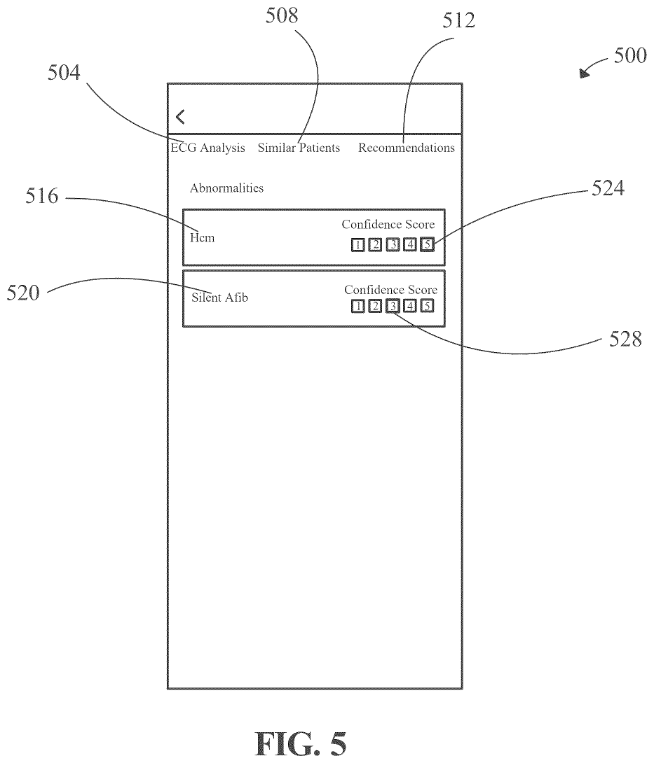

Referring now to FIG. 5, an exemplary embodiment of a user interface 500 is provided. User interface 500 may include one or more interactive elements by which a user may select varying functions of apparatus 100, such as elements 504, 508, and 512. User interface 500 may include one or more elements of abnormality data such as abnormality data 516 and 520 and/or one or more abnormality datum confidence scores such as abnormality datum confidence scores 524 and 528 associated with such abnormality data.

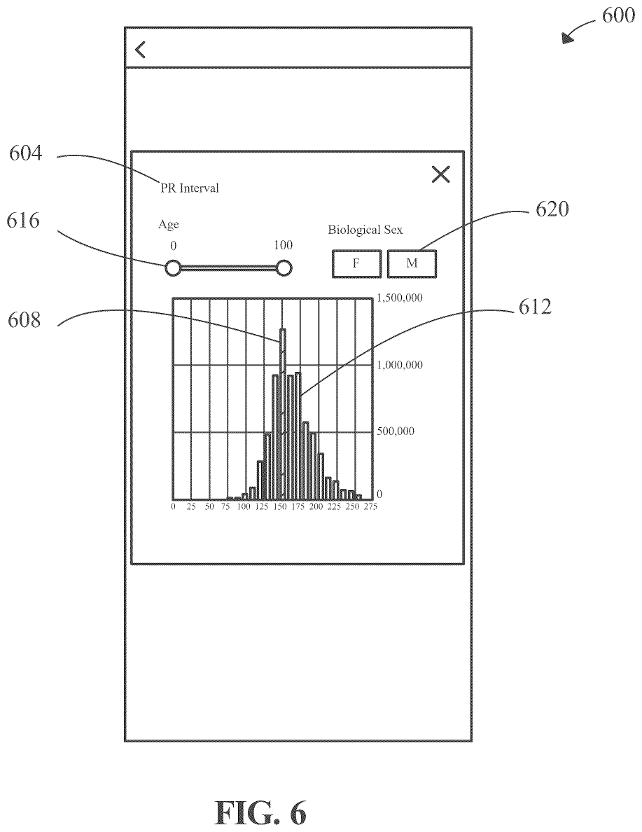

Referring now to FIG. 6, an exemplary embodiment of a user interface 600 is provided. User interface 600 may include an identifier 604 of a signal metric such as a name. User interface 600 may include signal metric position 608. User interface 600 may include data 612 of retrospective users as described with reference to FIG. 1. User interface 600 may include one or more sets of interactable features such as 616 and 620. Such sets of interactable features may be used to determine population restrictions. In some embodiments, a population restriction may be identified, and a population which a user's signal metric is compared to may be determined according to a population restriction. As used herein, a “population restriction” is a data structure setting a boundary on individuals to be considered members of a population. In non-limiting examples, population restrictions may include a limitation that members of a population be male, and a limitation that members of a population be under 25 years old. For example, set of interactable features 616 may restrict a population by age and set of interactable features 616 may restrict a population by biological sex.

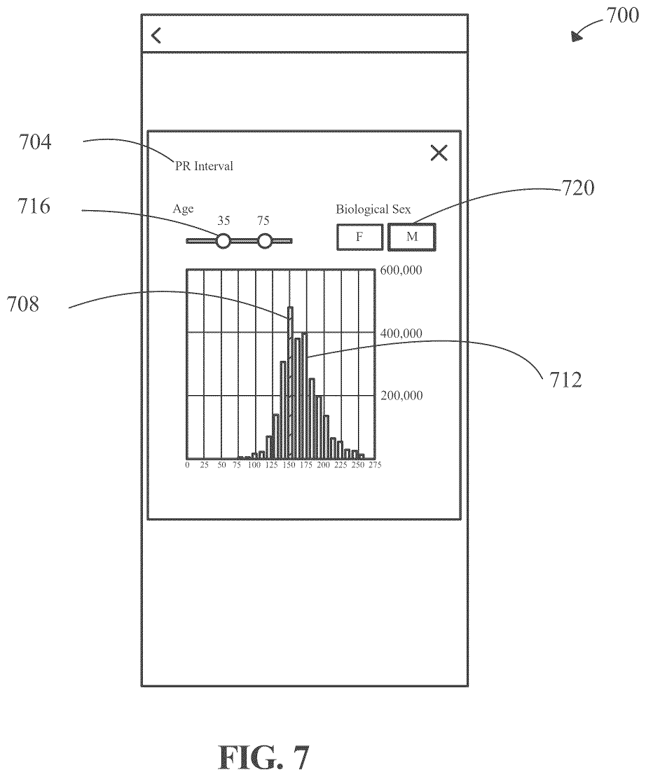

Referring now to FIG. 7, an exemplary embodiment of a user interface 700 is provided. User interface 700 may include an identifier 704 of a signal metric such as a name. User interface 700 may include signal metric position 708. User interface 700 may include data 712 of other members of a population as described with reference to FIG. 1. User interface 700 may include one or more sets of interactable features such as 716 and 720. Such sets of interactable features may be used to determine population restrictions as described with reference to FIG. 1. For example, set of interactable features 716 may restrict a population by age and set of interactable features 716 may restrict a population by biological sex.

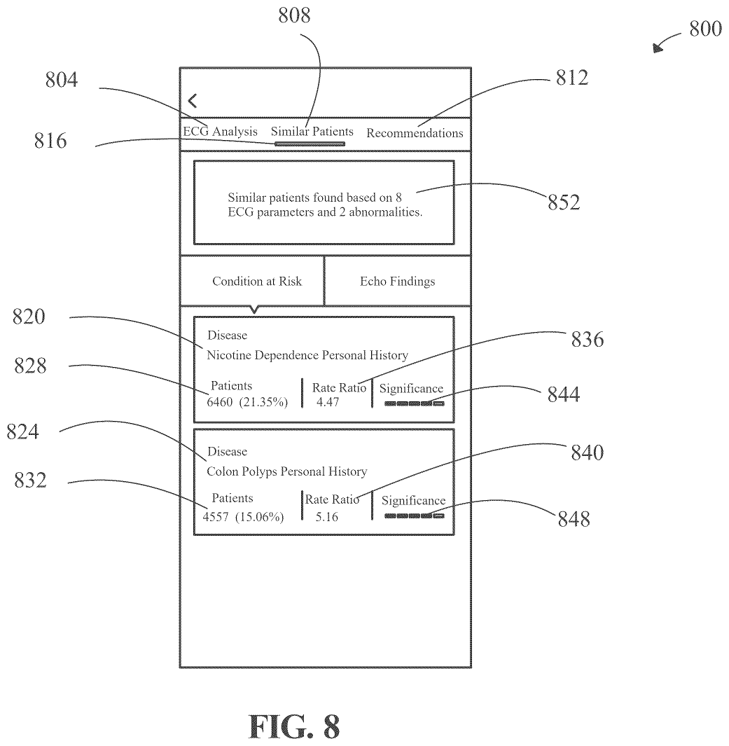

Referring now to FIG. 8, an exemplary embodiment of a user interface 800 is provided. User interface 800 may include one or more interactive elements by which a user may select varying functions of apparatus 100, such as elements 804, 808, and 812. User interface 800 may include an element which indicates a function currently selected, such as element 816. User interface 800 may include one or more elements of abnormality data 820 and 824. User interface 800 may include descriptions of metrics associated generation of abnormality data 820 and 824 based on similarity of one or more signal metrics with medical data of other patients. For example, user interface may include descriptions of metrics and cohort(s) 828, 832 describing numbers and percentages of similar patients (as determined with reference to FIG. 1), 836 and 840 describing rate ratios, 844 and 848 (describing degrees of significance of abnormality data). A rate ratio may refer to the likely of user being classified to system based on their data such as ECG data and vice versa. In some embodiments, the rate ratio may indicate a mortality rate for users classified under a specific symptom, disease, and the like. User interface 800 may include a description 852 which may, for example, provide a broad overview of contents of a page of user interface 800.

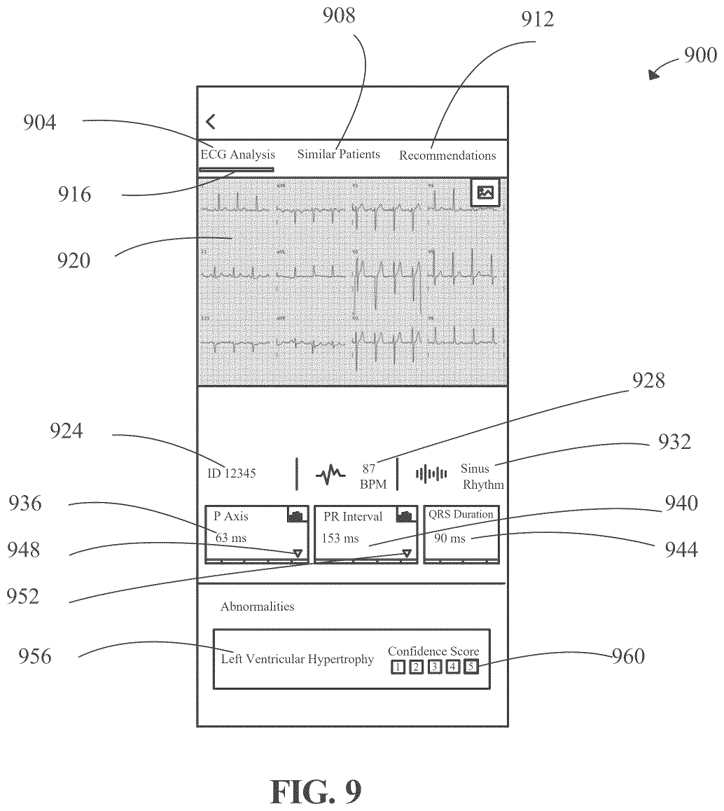

Referring now to FIG. 9, an exemplary embodiment of a user interface 900 is provided. User interface 900 may include one or more interactive elements by which a user may select varying functions of apparatus 100, such as elements 904, 908, and 912. User interface 900 may include an element which indicates a function currently selected, such as element 916. User interface 900 may include augmented image 920. In some embodiments, apparatus 100 may generate an augmented image as a function of image base user data using a trained augmented image machine learning model. In some embodiments, apparatus 100 may train an augmented image machine learning model by receiving raw data, generating a direct data digital image from the raw data, printing a physical image as a function of the raw data, generating a first scanned digital image by capturing an image of the physical image using a camera, and, using the direct data digital image and the first scanned digital image to train a machine learning model to generate a transformed digital image from a second scanned digital image. Such raw data may include, for example, voltage time series data received from a set of electrodes which measures electrical activity of the heart. Such direct data digital image may include, for example, a digital image plotting the raw data over time. Such direct data digital image and first scanned digital image may form a pair to be used as part of a training data set. Many such data pairs may be collected based on data, such as ECG data, of a variety of subjects. Such data pairs may make up a training dataset which is used to train augmented image machine learning model. Augmented image machine learning model may be trained such that it accepts as an input a scanned digital image (such as a picture of a paper ECG) and outputs an augmented image. An augmented image may be generated using a device and/or process disclosed in U.S. patent application Ser. No. 18/652,364 (having attorney docket number 1518-124USU1), filed on May 1, 2024, and titled “APPARATUS AND METHOD FOR TRAINING A MACHINE LEARNING MODEL TO AUGMENT SIGNAL DATA AND IMAGE DATA,” the entirety of which is hereby incorporated by reference.

Still referring to FIG. 9, image 920 may include a raw image and/or an image which has undergone one or more processing steps as described above. User interface 900 may include an identification number 924 for a particular signal and/or image. User interface 900 may include one or more signal metrics, such as signal metrics 928, 932, 936, 940, and 944. User interface 900 may include one or more signal metric positions, such as signal metric positions 948 and 952. User interface 900 may include one or more elements of abnormality data such as abnormality datum 956 and/or one or more abnormality datum confidence scores such as abnormality datum confidence score 960 associated with such abnormality data.

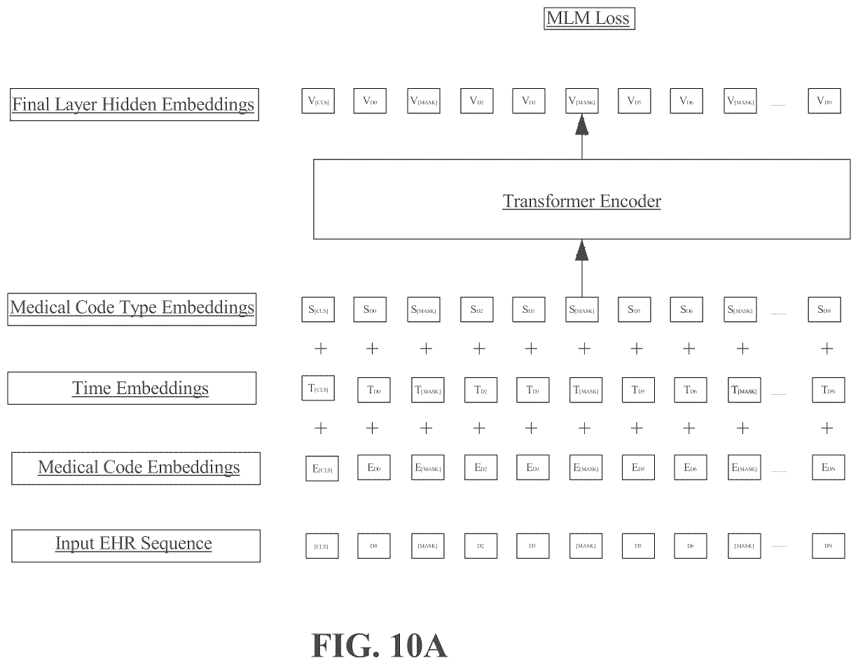

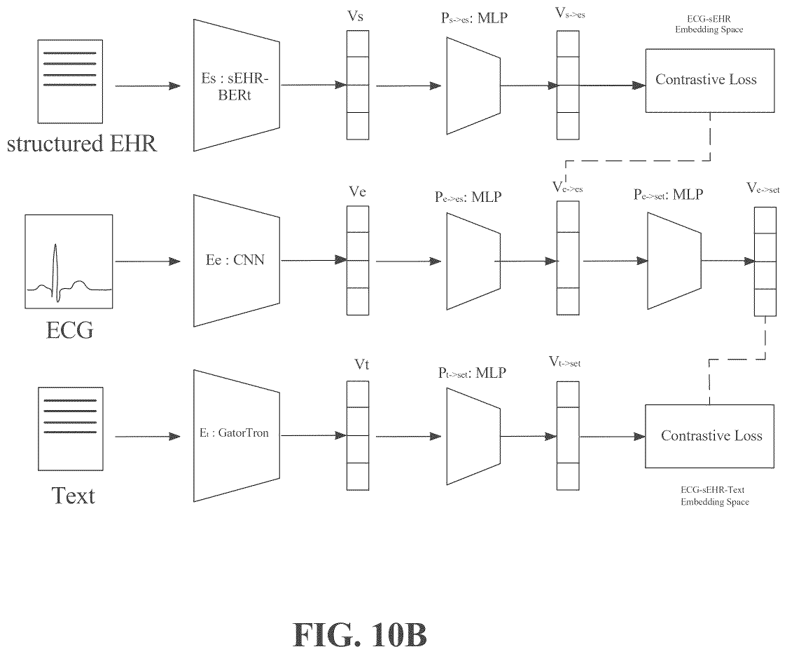

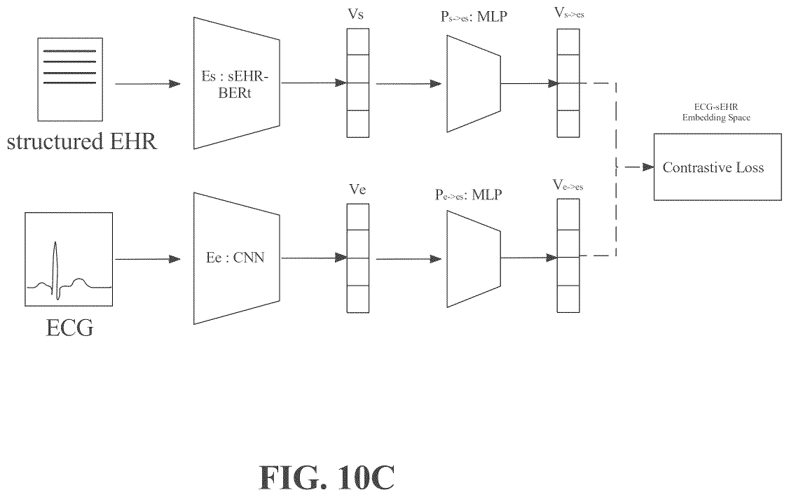

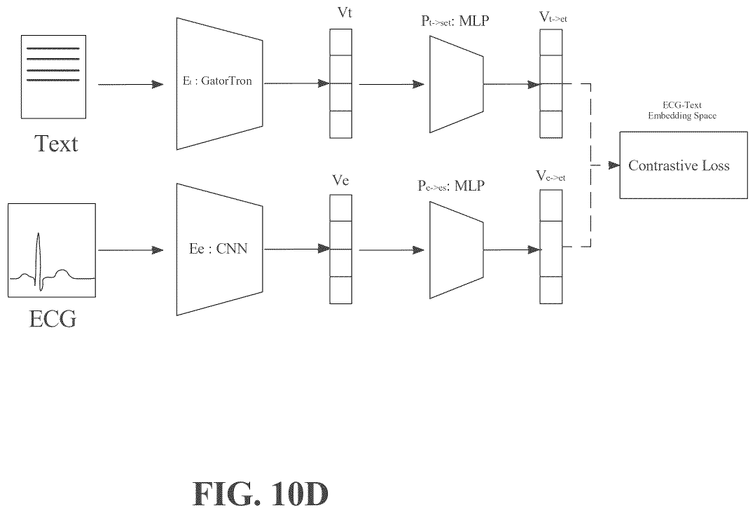

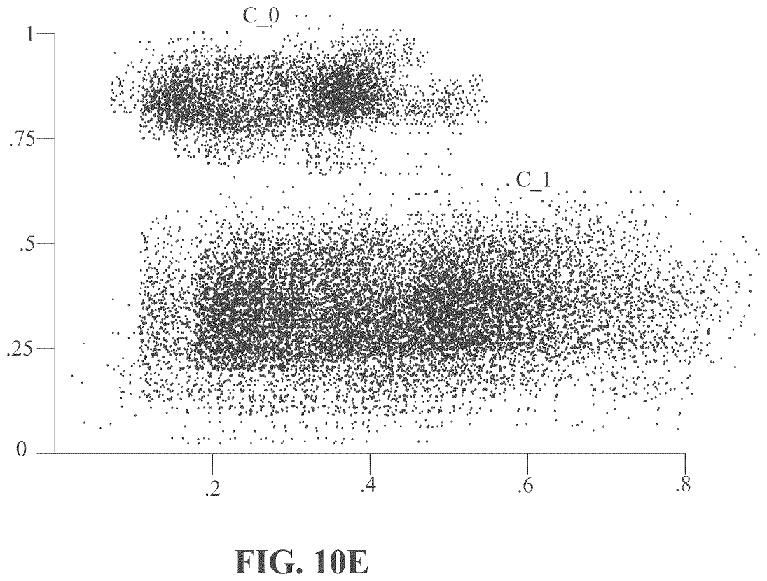

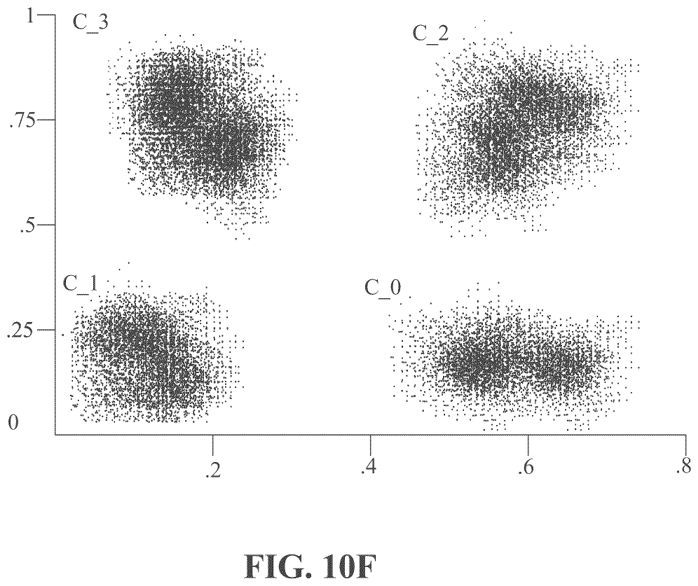

Now referring to FIGS. 10A-F, an exemplary embodiment of a plurality of models, such as one or models usable as a representation machine-learning model as described above, and which may be used to generate each of representation of the plurality of representations, is illustrated. Processor 104 may take a plurality of ICD diagnoses codes, ICD procedure codes and medication prescriptions that have an association with at least 50 patients and are considered in the vocabulary. Since ICD-9 codes are different from ICD-10 codes, but the underlying text descriptions are similar, processor 104 may do a mapping from ICD-9 to ICD-10 to maintain the same phenotypic information. Finally, ICD-10 diagnoses codes may be shortened to three characters as keeping four or more characters provides little to no extra information for large scale pre-training. A vocabulary of size 28593 is constructed based on medication prescriptions, ICD-10 procedure codes and shortened ICD-10 diagnoses codes. To create structured EHR sequence for structured EHR-BERT model pre-training processor 104 may randomly select one sequence of consecutive medical codes from a given patient's timeline.

With continued reference to FIG. 10B, the first representations may be generated using MultiModal Versatile Networks (MMV), which apply contrastive learning to video, audio, text multi-modal data under the assumption that the video and audio modalities are more granular than the text modality. MMV assumes that applying contrastive loss in shared embedding space does not maintain the specificities of each domain, so two embedding spaces are learned i.e. a fine-grained space where video and audio are matched, and coarse-grained space where text is matched with video and audio domains.

With continued reference to FIG. 10B, in some cases, the first representations may not exhibit the same level of granularity as the third representation and the second representation. The first representations within a given time window surrounding the ECG signals 108 acquisition may have their timestamps rounded or trimmed based on the input length accepted by the corresponding encoders. This may be the cause of the different granularity of information between the third representation and the second representation. The MMV may be used to compare ECG signal with structured EHR in fine-grained joint third representation embedding space Ωₑₛ and first representations in coarse-grained joint first representations Ωₛₑₜ.

With continued reference to FIG. 10B, a data set may include, S=Xₛ×Xₑ×Xₜ consisting of triplets

$$\{ ( {X}_{s}^{i} , {X}_{e}^{i} , {X}_{t}^{i} ) \} Mi = 1$$

where

$${X}_{s}^{i}$$

Is the structured EHR sequence of the i-th sample,

$${X}_{e}^{i}$$

is the ECG waveform of the i-th sample, and

$${X}_{t}^{i}$$

is the text sequence of the i-th sample, M is the total number of samples in the training set. Xₛ, Xₑ, and Xₜ denote the domain of the structured EHR, ECG and Text respectively.

With continued reference to FIG. 10B, in a non-limiting example, let Eₘ:Xₘ→R^{dm} be a parameterized model mapping from modality m to a modality specific embedding of dimension dm, where m can be s, e, t for structured EHR, ECG and Text respectively. Let Ωₛ be a shared space between different modalities where modality specific representations are projected into to maximize or minimize the alignment between different modalities using the contrastive loss objective. To obtain the modality specific representations processor 104 may use any suitable neural network architecture, such as without limitation a residual neural network (ResNet) architecture, other deep learning network architecture, and/or recurrent neural network architecture, customized to one dimension for the ECG encoder, such as without limitation a structured EHR-BERT encoder as described above for the structured EHR modality, and GatorTron encoder for the Text modality. Modality specific representations may be projected into shared space using a two layered fully connected network. In another example, let P_{m→s}: R^{dm}→R^{ds} be a projection network mapping from representation of modality m to representation in shared space Ωₛ. Processor 104 may apply a contrastive loss between ECG and structured EHR in ECG-structured EHR joint embedding space, and contrastive loss between ECG and Text in structured EHR-ECG-Text joint embedding space so that granularity is maintained.

With continued reference to FIG. 10B, In another embodiment,

$${V}_{m}^{i}$$

may represent a representation obtained by passing

$${X}_{m}^{i}$$

into modality specific encoder Eₘ,

$${V}_{m - s}^{i}$$

may represent a representation of Xₘ in the shared space Ωₛ obtained by passing

$${V}_{m}^{i}$$

into projection network P_{m→s}, Then, in a non-limiting example:

$${v}_{e}^{i} = {E}_{e} ( {x}_{e}^{i} )$$

$${v}_{s}^{i} = {E}_{s} ( {x}_{s}^{i} )$$

$${v}_{t}^{i} = {E}_{e} ( {x}_{t}^{i} )$$

$${v}_{e - es}^{i} = {P}_{e \to es} ( {v}_{e}^{i} )$$

$${v}_{s - es}^{i} = {P}_{s \to es} ( {v}_{s}^{i} )$$

$${v}_{e - set}^{i} = {P}_{e \to set} ( {v}_{e - es}^{i} )$$

$${v}_{t - set}^{i} = {P}_{e \to es} ( {v}_{t}^{i} )$$

Processor 104 may assume that all the above representations are l₂ normalized. Processor 104 may define a metric of similarity, such as without limitation cosine similarity s(x, y) between two l₂ normalized vectors, x, y∈R^{d} as:

$$s ( x , y ) = {x}^{T} y$$

In a non-limiting example, in a given minibatch of size N, let τ∈R⁺ be a temperature parameter, Lₑₛ be a contrastive loss between ECG and structured EHR,

$${L}_{i}^{e \to s}$$

be a contrastive loss directed from ECG to structured EHR, and

$${L}_{i}^{s \to e}$$

be a contrastive loss directed from structured EHR to ECG. Then,

$${L}_{i}^{e \to s} = - log \frac{exp ( s ( {v}_{e - es}^{i} , {v}_{s - es}^{i} ) / T )}{{\sum }_{K = 1}^{N} exp ( s ( {v}_{e - es}^{i} , {v}_{s - es}^{k} ) / T )}$$

$${L}_{i}^{s \to e} = - log \frac{exp ( s ( {v}_{s - es}^{i} , {v}_{e - es}^{i} ) / T )}{{\sum }_{K = 1}^{N} exp ( s ( {v}_{e - es}^{i} , {v}_{e - es}^{k} ) / T )}$$

$${L}_{es} = - \frac{1}{n} {\sum }_{i = 1}^{N} ( {λ}_{es} {L}_{i}^{e \to s} + ( 1 - {λ}_{es} ) {L}_{i}^{s \to e} )$$

Let Lₑₜ be the contrastive loss between ECG and Text,

$${L}_{i}^{e \to t}$$

be the contrastive loss directed from ECG to Text,

$${L}_{i}^{t \to t}$$

be the contrastive loss directed from Text to ECG, then:

$${L}_{i}^{e \to t} = - log \frac{exp ( s ( {v}_{e - set}^{i} , {v}_{t - set}^{i} ) / T )}{{\sum }_{K = 1}^{N} exp ( s ( {v}_{e - set}^{i} , {v}_{t - set}^{k} ) / T )}$$

$${L}_{i}^{t \to e} = - log \frac{exp ( s ( {v}_{t - set}^{i} , {v}_{t - set}^{i} ) / T )}{{\sum }_{K = 1}^{N} exp ( s ( {v}_{t - set}^{i} , {v}_{e - set}^{k} ) / T )}$$

$${L}_{et} = \frac{1}{n} {\sum }_{i = 1}^{N} ( {λ}_{es} {L}_{i}^{e \to t} + ( 1 - t ) {L}_{i}^{t \to e} )$$

the combination of which gives the overall loss:

$$L = {L}_{es} + {L}_{et}$$

where λes and λet are tunable hyperparameters ∈[0, 1]

With continued reference to FIG. 10C, to create the (Xₑ, Xₛ) ECG-structured EHR pairs, processor 104 may select an ECG of a given patient, Xₑ, and consider all the ICD diagnoses codes, ICD procedure codes and medication prescriptions associated with that patient within a period of one year prior, and one year subsequent, to the acquisition timestamp of that ECG. The medical codes restricted to this time range are arranged sequentially to form the initial structured EHR input sequence to the structured EHR-BERT model. Processor may use a maximum sequence length of 200 medical codes as input to the structured EHR-BERT encoder. The initial structured EHR input sequence with zeros if the structured EHR sequence length may be less than 200 and trimmed it by considering the nearest 200 medical codes to ECG acquisition timestamp if the structured EHR sequence length is greater than 200, to get the final Xₛ.

In the ECG-structured EHR model, processor 104 pairs ECGs with structured EHR data and apply multi-modal contrastive learning in joint ECG-structured EHR embedding space Ωₛ, discussed in greater detail herein above.

$${v}_{e}^{i} = {E}_{e} ( {x}_{e}^{i} )$$

$${v}_{s}^{i} = {E}_{s} ( {x}_{s}^{i} )$$

$${v}_{e - es}^{i} = {P}_{e \to es} ( {v}_{e}^{i} )$$

$${v}_{s - es}^{i} = {P}_{s \to es} ( {v}_{s}^{i} )$$

Let Lₑₛ be the contrastive loss between ECG and structured EHR,

$${L}_{i}^{e \to s}$$

be the contrastive loss directed from ECG to structured EHR, and

$${L}_{i}^{s \to e}$$

be the contrastive loss directed from structured EHR to ECG. Then, the loss for the ECG-structured EHR model is given by:

$${L}_{es} = \frac{1}{n} {\sum }_{i = 1}^{N} ( {λ}_{es} {L}_{i}^{e \to s} + ( 1 - {λ}_{es} ) {L}_{i}^{s \to e} )$$

Contrastive loss between ECG and structured EHR may be applied in ECG-sEHT joint embedding space where:

$${L}_{i}^{e \to s} = - log \frac{exp ( s ( {v}_{e - es}^{i} , {v}_{s - es}^{i} ) / T )}{{\sum }_{K = 1}^{N} exp ( s ( {v}_{e - es}^{i} , {v}_{s - es}^{k} ) / T )}$$

$${L}_{i}^{s \to e} = - log \frac{exp ( s ( {v}_{s - es}^{i} , {v}_{e - es}^{i} ) / T )}{{\sum }_{K = 1}^{N} exp ( s ( {v}_{s - es}^{i} , {v}_{e - es}^{k} ) / T )}$$

For a selected ECG, Xₑ, processor 104 may form multiple ECG-text pairs as an intermediate step. Textual data may include patient notes. Processor 104 may choose the one report that is closest in time to the ECG acquisition date, and pair it with the ECG, i.e. processor 104 forms pairs

$$( {X}_{e} , {X}_{t}^{k} )$$

with

$${X}_{t}^{k}$$

coming from different sources (k=ECG reports, ECHO reports, clinical documents, etc.). When forming the

$${X}_{t}^{k} ,$$

processor 104 may only use reports that were produced within 30 days after the ECG acquisition timestamp, except in the case of entity concatenation (described below) where processor 104 evaluated at reports produced in a time interval of one year pre- and post- the ECG acquisition timestamp.

From each of these intermediary reports,

$${X}_{t}^{k} ,$$

processor 104 may engage in an entity selection process an entity is a keyword that is medically relevant to the ECG being studied. By detecting those sentences in the notes that contain an entity, which may be chosen from a predetermined list, processor 104 can eliminate training on irrelevant data and improve the speed and potentially the performance of the representations produced.

Processor 104 may remove those sentences from the closest intermediary reports,

$${X}_{t}^{k} ,$$

that do not contain an entity. This is followed by randomly selecting one of these truncated intermediary reports to pair with the ECG. In the concatenation experiment, processor 104 may remove those sentences from the closest intermediary reports that do not contain an entity, but then concatenate all the truncated intermediary reports to form a final note, Xₜ, to pair with the ECG. In the entity concatenation experiment, processor 104 may focus not the sentences containing entities, but only the entities themselves, to form the final note, Xₜ. With the two latter experiments, the concatenation follows a priority order in which ECG reports precede ECHO reports, which in turn precede clinical, microbiology, pathology, radiology, and surgical notes.

With continued reference to FIG. 10D, In the ECG-Text model, processor 104 may pair electrocardiogram signals with unstructured text data obtained from a variety of medical sources, including ECG reports, ECHO reports, pathology reports, radiology reports, microbiology reports and clinical documents. These are collectively referred to as patient notes in this work. Processor 104 may apply the contrastive learning between ECG and Text in joint ECG-Text Embedding space Ωₑₜ

$${v}_{e}^{i} = {E}_{e} ( {x}_{e}^{i} )$$

$${v}_{t}^{i} = {E}_{t} ( {x}_{t}^{i} )$$

$${v}_{e - et}^{i} = {P}_{e \to et} ( {v}_{e}^{i} )$$

$${v}_{s - et}^{i} = {P}_{s \to et} ( {v}_{t}^{i} )$$

In an embodiment, Lₑₜ be the contrastive loss between ECG and Text,

$${L}_{i}^{e \to t}$$

be the contrastive loss directed from ECG to Text, and

$${L}_{i}^{t \to e}$$

be the contrastive loss directed from Text to ECG. Then, the loss for the ECG-Text model is given by:

$${L}_{et} = \frac{1}{n} {\sum }_{i = 1}^{N} ( {λ}_{et} {L}_{i}^{e \to t} + ( 1 - {λ}_{et} ) {L}_{i}^{t \to e} )$$

$${L}_{i}^{s \to t} = - log \frac{exp ( s ( {v}_{e - et}^{i} , {v}_{t - et}^{i} ) / T )}{{\sum }_{K = 1}^{N} exp ( s ( {v}_{e - et}^{i} , {v}_{t - et}^{k} ) / T )}$$

Counts for ECGs and unique patients for each downstream task cohort.

$${L}_{i}^{t \to e} = - log \frac{exp ( s ( {v}_{t - et}^{i} , {v}_{e - et}^{i} ) / T )}{{\sum }_{K = 1}^{N} exp ( s ( {v}_{t - et}^{i} , {v}_{e - et}^{k} ) / T )}$$

With continued reference to FIGS. 10A-D, embedding/statistical representations learnt using a plurality of methods as described above may be evaluated on various downstream tasks. For example, processor 104 may use the representations obtained, such as ECG embeddings, as inputs to a logistic regression architecture to train various linear models for disease classification tasks. These diseases may include Atherosclerotic cardiovascular disease (ASCVD), Myocarditis, Pulmonary Hypertension (PH), Left ventricular ejection fraction (LVEF), Atrial fibrillation in Normal Sinus Rhythm (AFib in NSR). Processor 104 may compare the performance of the linear classification models against two baseline supervised learning models; the first may be a neural net trained from scratch using random initialization of its weights, while the second may be a large-scale multitask learning (‘MTL’) model, described below. Processor 104 may evaluate the performance (AUC) of various models, as described throughout this disclosure, on linear classification task trained across different disease cohorts and across different fractions of the training set, i.e. 1%, 10%, 100%.

With continued reference to FIGS. 10A-D, all models, across diseases, show improvement over the models trained from scratch with random initialization. The ECG-structured EHR model may be the overall best-performing model with a slight drop from large scale ECG-MTL model in case of LVEF and PH diseases. The difference between the ECG-structured EHR and ECG-Text models varies between 1-2%.

With continued reference to FIGS. 10A-D, in high data environments such as PH, LVEF and AFib in NSR, the drop in performance between a model trained on the full training data (100%) and ob 10% of the training data may not be too large. The large drop in performance on the 1% subset in diseases such as Myocarditis can be explained by the small cohort size of even the full training set.

With continued reference to FIGS. 10A-D, Using ECG representations obtained from ECG-structured EHR, processor 104 observes that linear classification models trained on 10% of training data across all diseases achieve performance comparable to or better than that of the random weight initialization model trained on the full training dataset, showing the effectiveness of the learned representations for label efficiency.

With continued reference to FIGS. 10A-D, Processor 104 may observe that the representations learned via self-supervised learning techniques help to better distinguish datum that comes from out of the distribution under consideration. Processor 104 may demonstrate this using representations obtained from our EHR model to distinguish between two disparate ECG datasets. Processor 104 may take the proprietary ECG pulmonary hypertension (PH) cohort as the ‘In Distribution’, and holter ECGS from the open-source St Petersburg INCART 12-lead Arrhythmia Database as the ‘Out Distribution’. Processor 104 may train a CNN network (PH model) in supervised setting on our PH training data to compare the performance of both the representations. Processor 104 may use three metrics to determine whether the data is in or out of distribution—the Relative Mahalanobis Distance (RMD), the Class Conditional Mahalanobis Distance (CCMD), and the Cleanlab Out of distribution. While Relative Mahalanobis Distance is based on mahalanobis distance of embeddings from nearest predicted class, clean lab uses a K-Nearest Neighbor based approach to distinguish In vs Out distribution samples. We use the representations from the EHR model and the PH model and show that that the rejection rate at different significance levels is much higher when the EHR model representations are used. The results may show that generic ECG representations are better at detecting out-of-distribution data than model specific representations.

With continued reference to FIGS. 10A-D, processor 104 may be configured to identify a quality of a plurality of representations. A plurality of representations is said to be good if it satisfies several criteria. The criteria may include expressiveness, abstraction and invariance, and disentanglement. Expressiveness may include the ability of the plurality of representations to represent a large number of input configurations. Abstraction and invariance may include the ability of the plurality of representations to encode high level information, and thus be invariant to small local variations of the data. Disentanglement may include the ability to learn all explanatory features while preserving orthogonality of distinct factors. The quality of a plurality of representations may be quantified by the improvement in performance it leads to in a downstream task, although there is often a trade-off between good performance over a wide range of tasks and excellent performance in a specialized task. If the representations are learned via a proxy task, such as similarity in contrastive learning, performance on the proxy task can serve as a metric for measuring quality of the representation.

With continued reference to FIGS. 10E-F, Representations can be used to better and more quickly cluster similar data samples together. We demonstrate this by comparing the clustering on the PH cohort obtained using the EHR model and the PCLR model representations. Processor 104 may a density based clustering algorithms, HDBSCAN, performed on the UMAP space of positive ECGs. The cluster strength for a cluster i, Cᵢ is given by:

$${C}_{i} = \frac{{P}_{i} log ( {P}_{i} + {N}_{i} )}{{P}_{i} + {N}_{i}}$$

where Pi is the count of positive ECGs in the ith cluster, and Ni is the count of negative ECGs in the ith cluster. The cluster strength value above quantifies the proportion of positives along with total count of ECGs in a particular cluster. To compare the clustering ability of two sets of embeddings, we use the overall cluster strength:

$$E C = \frac{{\sum }_{i} {C}_{i}}{{\sum }_{j} log ( {P}_{j} + {N}_{j} )}$$

FIGS. 10E-F show the results of clustering on the EHR model and PCLR model embeddings, respectively. The former has an overall cluster strength of 0.5149, while the PCLR embeddings cluster with an overall strength of 0.3125.

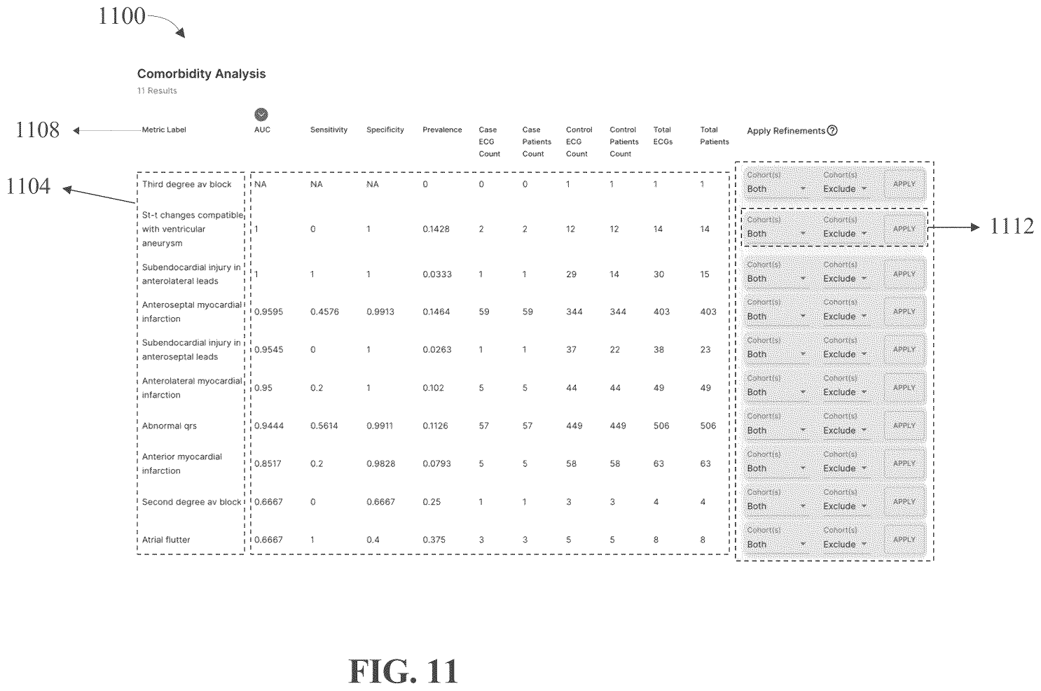

Referring now to FIG. 11, an exemplary embodiment of a user interface 1100 for displaying a comorbidity analysis is provided. A “comorbidity,” as used herein, refers to the simultaneous presence of two or more medical conditions or diseases 1104 in a user. Cohort A may be a cohort generated based on a query input, such as specify based on any records in EHR data including notes, ICD codes, and the like. In some embodiments, Cohort B may be a control group absent or excluding features of the query input. For example, Cohort B may include control set of ECGs. In some embodiments a third party, such as a health professional, may be able to use a preliminary cohort(s), as described in FIG. 1, as a control group (Cohort B). In other embodiments, Cohort A and Cohort B may be cohorts derived from a query input, AI derived criteria, expert/clinical derived criteria, or any other method of generating a cohort as described in FIG. 1. User interface 1100 may provide a comorbidity analysis as a function of a disease classification of a plurality of users/cohorts. For example, processor 104 may be classify users in cohort A & B to one or more medical conditions/diseases 1104 and the like using a condition evaluation model as disclosed in U.S. patent application Ser. No. 18/229,854 (having attorney docket number 1518-101USU1), filed on Aug. 3, 2023, and titled “APPARATUS AND METHOD FOR DETERMINING A PATIENT SURVIVAL PROFILE USING ARTIFICIAL INTELLIGENCE-ENABLED ELECTROCARDIOGRAM (ECG),” the entirety of which is hereby incorporated by reference. In some embodiments, user may use user interface 1100 to add inclusions or exclusions to the cohort, query input 120 and/or query criteria 124 disclosed with reference to FIG. 1.

Still referring to FIG. 11, user interface may break down the cohorts by medical conditions or diseases 1104, wherein a plurality of metric labels 1108 are displayed with each medical condition or disease grouping. Metric labels 1108 may include metrics for a classification model (e.g., a logistic regression classifier) trained on ECG representations, such as a condition evaluation model. For example, condition evaluation model may be trained on data correlating ECGs to the presence (or absence) of a condition. Metric labels 1108 may include AUC, sensitivity, specificity, case ECG count, case patients count, control ECG count, control patients count, total ECGs, and total patients. AUC (Area Under the Curve) refers to the area under the Receiver Operating Characteristic (ROC) curve. It's a performance measurement for the classification model at various threshold settings. AUC represents the degree of separability, indicating how well the model can distinguish between classes (e.g., patients with a condition vs. without it). An AUC of 1 may indicate a perfect prediction, while an AUC of 0.5 may suggest no discriminative power. Specificity measures the proportion of true negatives that are correctly identified by the model. For example, a high specificity means the test is good at ruling out the disease in patients who don't have it. Sensitivity measures the proportion of actual positives (e.g., patients with the condition) that are correctly identified. It reflects the test's ability to correctly detect patients who do have the condition. Case ECG Count refers to the number of ECGs that were classified as cases (patients with the condition) by the model. Case Patients Count is the number of patients classified as cases. It's important to differentiate this from the ECG count since one patient may have multiple ECGs. Control ECG Count refers to the number of ECGs that were classified as controls (patients without the condition). Control Patients Count refers to the number of patients classified as controls, distinguishing from the ECG count for similar reasons mentioned above. Total ECGs is total number of ECGs used in the analysis, combining both cases and controls. Total Patients is the total number of patients involved in the classification, summing both case patients and control patients.

Still referring to FIG. 11, user interface 11 may include widget(s) 1112 to enable a user/third party to control what action may performed such as including or excluding cohorts and the like for the analysis by medical condition/disease grouping. For example, a third party, such as health care professional, may refine both or either cohorts A and B to include or exclude a patients/users based on the medical condition/disease.

Still referring to FIG. 11, for each comorbidity D there may be a specific list of cohorts C_1D, C_2D, C_3D, and the like, that is either searchable within the nference platform apriori or that the physician may create on the fly such that C_1D, C_2D, C_3D, and the like, are all clinically relevant groups of patients. For example, this may include groups of patients that are clinically relevant to the diagnosis D being considered. In some embodiments, the physician may, in parallel, query for enrichments from clinically relevant cohorts for many indications/comorbidities/diagnoses all at once. In some embodiments, physician may also ask for AI as well as non-AI neighborhoods within each such clinically relevant cohort, for each such indication/comorbidity/diagnosis they have in mind. In some embodiment, the physician may perform differential diagnoses by studying all of the enrichments. There may be multiple methods to rank and statistically score different diagnoses based on enrichments-based AI and non-AI based neighborhoods and cohorts.

With reference to FIGS. 1, 3, 8 and 11, an exemplary scenario in clinical context may include that patient's ECG (or another modality, such as CT scans, X-rays, echocardiograms, EHR data, and the like) shows an abnormality such as premature ventricular contraction (PVC). The differential diagnosis for a PVC has several options, some of which are considered safe in the short term, and some which need immediate care. This could technically be any abnormality or pattern seen on an ECG (or another modality, such as CT scans, X-rays, echocardiograms, EHR data, and the like) that has wide-ranging differential diagnosis with different clinical management pathways. While the naïve way to determine the appropriate clinical course of action may be to carry out additional tests such as blood work or echocardiograms, other imaging test, the physician can use the “similar patients” feature to show similar patients (e.g., see 308 in FIG. 3). Sometimes the course of action may just be a referral with an expert cardiologist or electrophysiologist for a second opinion. The “similar patients” feature may show ECGs (or another modality, such as CT scans, X-rays, echocardiograms, EHR data, and the like). that have had similar presentation as the patient being examined. Physician may then, using a user interface, such as user interface see various characteristics of the patient cohort that had similar ECGs (See, e.g., user interface 800, described with reference to FIG. 8). This may include common Echo findings, survival curves, diagnosis, drugs/treatments and other clinical variable enrichments. In some embodiments, based on the above data, the physician may be able to make a sound decision on patient management that would have otherwise needed action.

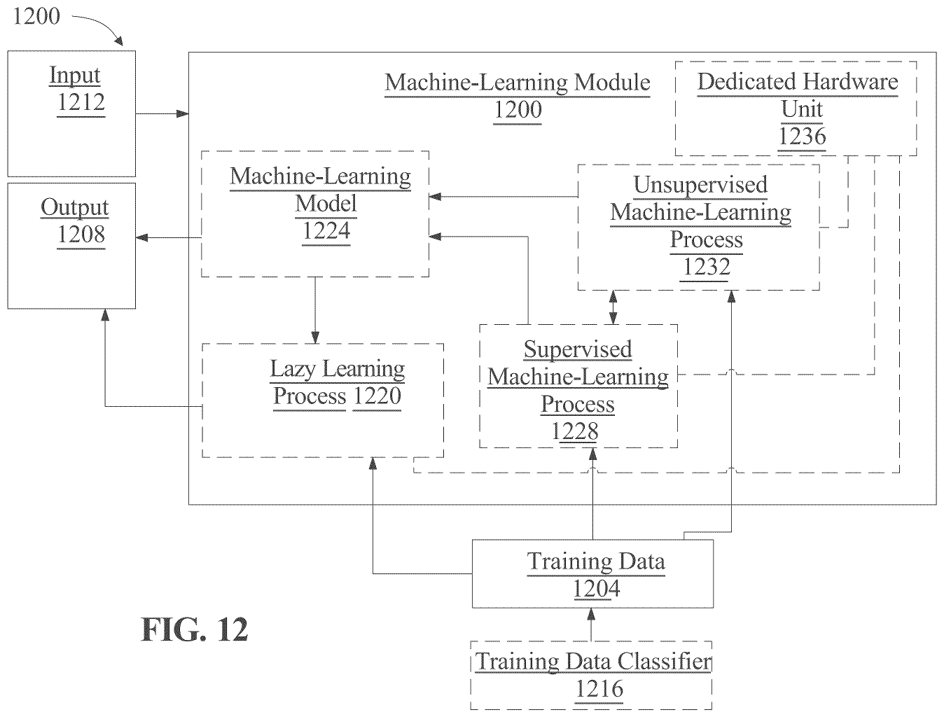

Referring now to FIG. 12, an exemplary embodiment of a machine-learning module 1200 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 1204 to generate an algorithm instantiated in hardware or software logic, data structures, and/or functions that will be performed by a computing device/module to produce outputs 1208 given data provided as inputs 1212; this is in contrast to a non-machine learning software program where the commands to be executed are determined in advance by a user and written in a programming language.

Still referring to FIG. 12, “training data,” as used herein, is data containing correlations that a machine-learning process may use to model relationships between two or more categories of data elements. For instance, and without limitation, training data 1204 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 1204 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 1204 according to various correlations; correlations may indicate causative and/or predictive links between categories of data elements, which may be modeled as relationships such as mathematical relationships by machine-learning processes as described in further detail below. Training data 1204 may be formatted and/or organized by categories of data elements, for instance by associating data elements with one or more descriptors corresponding to categories of data elements. As a non-limiting example, training data 1204 may include data entered in standardized forms by persons or processes, such that entry of a given data element in a given field in a form may be mapped to one or more descriptors of categories. Elements in training data 1204 may be linked to descriptors of categories by tags, tokens, or other data elements; for instance, and without limitation, training data 1204 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 12, training data 1204 may include one or more elements that are not categorized; that is, training data 1204 may not be formatted or contain descriptors for some elements of data. Machine-learning algorithms and/or other processes may sort training data 1204 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 1204 to be made applicable for two or more distinct machine-learning algorithms as described in further detail below. Training data 1204 used by machine-learning module 1200 may correlate any input data as described in this disclosure to any output data as described in this disclosure.

Further referring to FIG. 12, training data may be filtered, sorted, and/or selected using one or more supervised and/or unsupervised machine-learning processes and/or models as described in further detail below; such models may include without limitation a training data classifier 1216. Training data classifier 1216 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 1200 may generate a classifier using a classification algorithm, defined as a processes whereby a computing device and/or any module and/or component operating thereon derives a classifier from training data 1204. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 1216 may classify elements of training data to a diagnosis cohort, medical history cohort, symptom cohort, modality cohort and the like.

Still referring to FIG. 12, Computing device may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device may then calculate a likelihood table by calculating probabilities of different data entries and classification labels. Computing device may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 12, Computing device may be configured to generate a classifier using a K-nearest neighbors (KNN) algorithm. A “K-nearest neighbors algorithm” as used in this disclosure, includes a classification method that utilizes feature similarity to analyze how closely out-of-sample- features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 12, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 7, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm:

$$l = \sqrt{{\sum }_{i = 0}^{n} {a}_{i}^{2}} ,$$

where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 12, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 12, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 12, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 12, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity, and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 12, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 70 pixels, however a desired number of pixels may be 128. Processor may interpolate the low pixel count image to convert the 70 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 12, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 12, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 12, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset

$${X}_{m ax} : {X}_{n ew} = \frac{X - {X}_{m i n}}{{X}_{m ax} - {X}_{m i n}} .$$

Feature scaling may include mean normalization, which involves use of a mean value of a set and/or subset of values, Xₘₑₐₙ with maximum and minimum values:

$${X}_{n ew} = \frac{X - {X}_{mean}}{{X}_{m ax} - {X}_{m i n}} .$$

Feature scaling may include standardization, where a difference between X and Xₘₑₐₙ is divided by a standard deviation σ of a set or subset of values:

$${X}_{new} = \frac{X - {X}_{mean}}{σ} .$$

Scaling may be performed using a median value of a a set or subset X_{median} and/or interquartile range (IQR), which represents the difference between the 25^{th} percentile value and the 50^{th} percentile value (or closest values thereto by a rounding protocol), such as:

$${X}_{new} = \frac{X - {X}_{median}}{IQR} .$$

Persons skilled in the art, upon reviewing the entirety of this disclosure, will be aware of various alternative or additional approaches that may be used for feature scaling.

Further referring to FIG. 12, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 12, machine-learning module 1200 may be configured to perform a lazy-learning process 1220 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 1204. Heuristic may include selecting some number of highest-ranking associations and/or training data 1204 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 12, machine-learning processes as described in this disclosure may be used to generate machine-learning models 1224. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 1224 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 1224 may be generated by creating an artificial neural network, such as a convolutional neural network comprising an input layer of nodes, one or more intermediate layers, and an output layer of nodes. Connections between nodes may be created via the process of “training” the network, in which elements from a training data 1204 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 12, machine-learning algorithms may include at least a supervised machine-learning process 1228. At least a supervised machine-learning process 1228, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include inputs as described in this disclosure as inputs, outputs as described in this disclosure as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 1204. Persons skilled in the art, upon reviewing the entirety of this disclosure, will be aware of various possible variations of at least a supervised machine-learning process 1228 that may be used to determine relation between inputs and outputs. Supervised machine-learning processes may include classification algorithms as defined above.

With further reference to FIG. 12, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 12, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 12, machine learning processes may include at least an unsupervised machine-learning processes 1232. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 1232 may not require a response variable; unsupervised processes 1232 may be used to find interesting patterns and/or inferences between variables, to determine a degree of correlation between two or more variables, or the like.

Still referring to FIG. 12, machine-learning module 1200 may be designed and configured to create a machine-learning model 1224 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 12, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 12, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 12, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 12, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 12, one or more processes or algorithms described above may be performed by at least a dedicated hardware unit 1236. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 1236 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 1236 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 1236 to perform one or more operations described herein, such as evaluation of model and/or algorithm outputs, one-time or iterative updates to parameters, coefficients, weights, and/or biases, and/or any other operations such as vector and/or matrix operations as described in this disclosure.

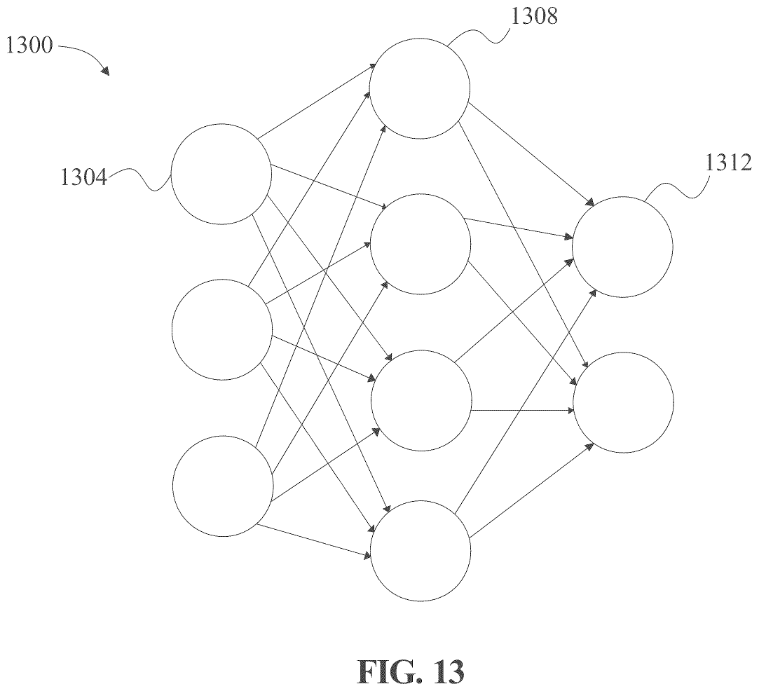

Referring now to FIG. 13, an exemplary embodiment of neural network 1300 is illustrated. A neural network 1300 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 1304, one or more intermediate layers 1308, and an output layer of nodes 1312. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

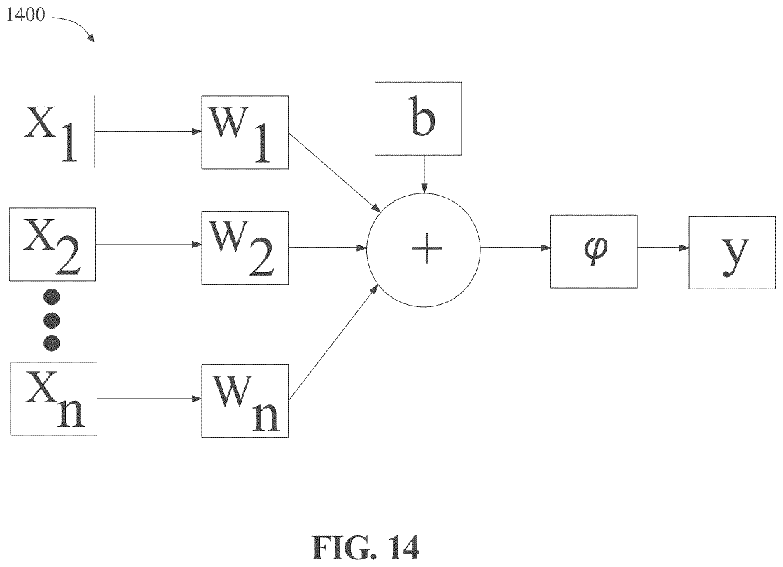

Referring now to FIG. 14, an exemplary embodiment of a node 1400 of a neural network is illustrated. A node may include, without limitation a plurality of inputs xᵢ that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tanh (hyperbolic tangent) function, of the form

$$\frac{{e}^{x} - {e}^{- x}}{{e}^{x} + {e}^{- x}} ,$$

a tanh derivative function such as ƒ(x)=tanh²(x), a rectified linear unit function such as ƒ(x)=max(0, x), a “leaky” and/or “parametric” rectified linear unit function such as ƒ(x)=max(ax, x) for some a, an exponential linear units function such as

$$f ( x ) = \{ \begin{matrix}x for x \geq 0 \\ α ( {e}^{x} - 1 ) for x < 0\end{matrix}$$

for some value of α (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as ƒ(x)=x*sigmoid(x), a Gaussian error linear unit function such as

$$f ( x ) = a ( 1 + tanh ( \sqrt{2 / π} ( x + {bx}^{r} ) ) )$$

for some values of a, b, and r, and/or a scaled exponential linear unit function such as

$$f ( x ) = λ \{ \begin{matrix}α ( {e}^{x} - 1 ) for x < 0 \\ x for x \geq 0\end{matrix} .$$

Fundamentally, there is no limit to the nature of functions of inputs xᵢ that may be used as activation functions. As a non-limiting and illustrative example, node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

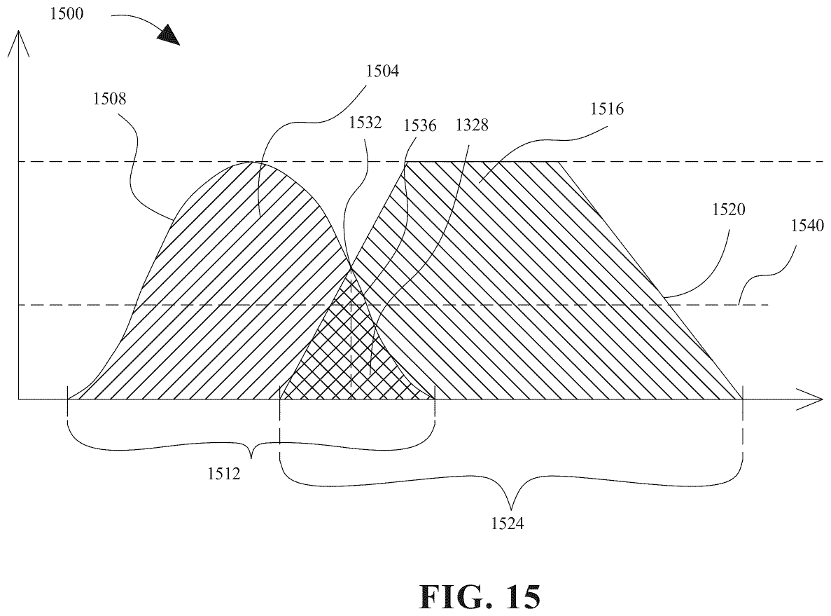

Referring to FIG. 15, an exemplary embodiment of fuzzy set comparison 1500 is illustrated. A first fuzzy set 1504 may be represented, without limitation, according to a first membership function 1508 representing a probability that an input falling on a first range of values 1512 is a member of the first fuzzy set 1504, where the first membership function 1508 has values on a range of probabilities such as without limitation the interval [0,1], and an area beneath the first membership function 1508 may represent a set of values within first fuzzy set 1504. Although first range of values 1512 is illustrated for clarity in this exemplary depiction as a range on a single number line or axis, first range of values 1512 may be defined on two or more dimensions, representing, for instance, a Cartesian product between a plurality of ranges, curves, axes, spaces, dimensions, or the like. First membership function 1508 may include any suitable function mapping first range 1512 to a probability interval, including without limitation a triangular function defined by two linear elements such as line segments or planes that intersect at or below the top of the probability interval. As a non-limiting example, triangular membership function may be defined as:

$$y ( x , a , b , c ) = \{ \begin{matrix}0 , for x > c and x < a \\ \frac{x - a}{b - a} , for a \leq x < b \\ \frac{c - x}{c - b} , if b < x \leq c\end{matrix}$$

a trapezoidal membership function may be defined as:

$$y ( x , a , b , c , d ) = max ( min ( \frac{x - a}{b - a} , 1 , \frac{d - x}{d - c} ) , 0 )$$

a sigmoidal function may be defined as:

$$y ( x , a , c ) = \frac{1}{1 - {e}^{- a ( x - c )}}$$

a Gaussian membership function may be defined as:

$$y ( x , c , σ ) = {e}^{- \frac{1}{2} {( \frac{x - c}{σ} )}^{2}}$$

and a bell membership function may be defined as:

$$y ( x , a , b , c ) = {[ 1 + {\vert \frac{x - c}{a} \vert}^{2 b} ]}^{- 1}$$

Persons skilled in the art, upon reviewing the entirety of this disclosure, will be aware of various alternative or additional membership functions that may be used consistently with this disclosure.

Still referring to FIG. 15, first fuzzy set 1504 may represent any value or combination of values as described above, including output from one or more machine-learning models, user data, and a predetermined class, such as without limitation of, a retrospective patient cohort. A second fuzzy set 1516, which may represent any value which may be represented by first fuzzy set 1504, may be defined by a second membership function 1520 on a second range 1524; second range 1524 may be identical and/or overlap with first range 1512 and/or may be combined with first range via Cartesian product or the like to generate a mapping permitting evaluation overlap of first fuzzy set 1504 and second fuzzy set 1516. Where first fuzzy set 1504 and second fuzzy set 1516 have a region 1528 that overlaps, first membership function 1508 and second membership function 1520 may intersect at a point 1532 representing a probability, as defined on probability interval, of a match between first fuzzy set 1504 and second fuzzy set 1516. Alternatively or additionally, a single value of first and/or second fuzzy set may be located at a locus 1536 on first range 1512 and/or second range 1524, where a probability of membership may be taken by evaluation of first membership function 1508 and/or second membership function 1520 at that range point. A probability at 1528 and/or 1532 may be compared to a threshold 1540 to determine whether a positive match is indicated. Threshold 1540 may, in a non-limiting example, represent a degree of match between first fuzzy set 1504 and second fuzzy set 1516, and/or single values therein with each other or with either set, which is sufficient for purposes of the matching process; for instance, threshold may indicate a sufficient degree of overlap between an output from one or more machine-learning models and/or user data and a predetermined class, such as without limitation a retrospective patient cohort categorization, for combination to occur as described above. Alternatively or additionally, each threshold may be tuned by a machine-learning and/or statistical process, for instance and without limitation as described in further detail below.

Further referring to FIG. 15, in an embodiment, a degree of match between fuzzy sets may be used to classify user data with retrospective patient cohort. For instance, if a retrospective patient cohort has a fuzzy set matching user data fuzzy set by having a degree of overlap exceeding a threshold, processor 104 may classify the user data as belonging to the retrospective patient cohort categorization. Where multiple fuzzy matches are performed, degrees of match for each respective fuzzy set may be computed and aggregated through, for instance, addition, averaging, or the like, to determine an overall degree of match.

Still referring to FIG. 15, in an embodiment, user data may be compared to multiple retrospective patient cohort categorization fuzzy sets. For instance, user data may be represented by a fuzzy set that is compared to each of the multiple retrospective patient cohort categorization fuzzy sets; and a degree of overlap exceeding a threshold between the user data fuzzy set and any of the multiple retrospective patient cohort categorization fuzzy sets may cause processor 104 to classify the user data as belonging to retrospective patient cohort categorization. For instance, in one embodiment there may be two retrospective patient cohort categorization fuzzy sets, representing respectively a first retrospective patient cohort categorization and a second retrospective patient cohort categorization. First retrospective patient cohort categorization may have a first fuzzy set; Second retrospective patient cohort categorization may have a second fuzzy set; and user data may have a user data fuzzy set. processor 104, for example, may compare a user data fuzzy set with each of first retrospective patient cohort categorization fuzzy set and second retrospective patient cohort categorization fuzzy set, as described above, and classify a user data to either, both, or neither of retrospective patient cohort categorization or retrospective patient cohort categorization. Machine-learning methods as described throughout may, in a non-limiting example, generate coefficients used in fuzzy set equations as described above, such as without limitation x, c, and σ of a Gaussian set as described above, as outputs of machine-learning methods. Likewise, user data may be used indirectly to determine a fuzzy set, as user data fuzzy set may be derived from outputs of one or more machine-learning models that take the user data directly or indirectly as inputs.

Still referring to FIG. 15, a computing device may use a logic comparison program, such as, but not limited to, a fuzzy logic model to determine a retrospective patient cohort response. An retrospective patient cohort response may include, but is not limited to, incompatible, compatible, and the like; each such retrospective patient cohort response may be represented as a value for a linguistic variable representing retrospective patient cohort response or in other words a fuzzy set as described above that corresponds to a degree of similarity as calculated using any statistical, machine-learning, or other method that may occur to a person skilled in the art upon reviewing the entirety of this disclosure. In other words, a given element of user data may have a first non-zero value for membership in a first linguistic variable value such as “compatible” and a second non-zero value for membership in a second linguistic variable value such as “incompatible” In some embodiments, determining a retrospective patient cohort categorization may include using a linear regression model. A linear regression model may include a machine learning model. A linear regression model may be configured to map data of user data, such as degree of similarity to one or more retrospective patient cohort parameters. A linear regression model may be trained using a machine learning process. A linear regression model may map statistics such as, but not limited to, quality of user data compatibility. In some embodiments, determining an retrospective patient cohort of user data may include using a retrospective patient cohort classification model. A retrospective patient cohort classification model may be configured to input collected data and cluster data to a centroid based on, but not limited to, frequency of appearance, linguistic indicators of quality, and the like. Centroids may include scores assigned to them such that quality of compatibility of user data may each be assigned a score. In some embodiments retrospective patient cohort classification model may include a K-means clustering model. In some embodiments, retrospective patient cohort classification model may include a particle swarm optimization model. In some embodiments, determining the retrospective patient cohort of user data may include using a fuzzy inference engine. A fuzzy inference engine may be configured to map one or more user data data elements using fuzzy logic. In some embodiments, user data may be arranged by a logic comparison program into retrospective patient cohort arrangement. A “retrospective patient cohort arrangement” as used in this disclosure is any grouping of objects and/or data based on skill level and/or output score. This step may be implemented as described above in FIGS. 1-14. Membership function coefficients and/or constants as described above may be tuned according to classification and/or clustering algorithms. For instance, and without limitation, a clustering algorithm may determine a Gaussian or other distribution of questions about a centroid corresponding to a given compatibility level, and an iterative or other method may be used to find a membership function, for any membership function type as described above, that minimizes an average error from the statistically determined distribution, such that, for instance, a triangular or Gaussian membership function about a centroid representing a center of the distribution that most closely matches the distribution. Error functions to be minimized, and/or methods of minimization, may be performed without limitation according to any error function and/or error function minimization process and/or method as described in this disclosure.

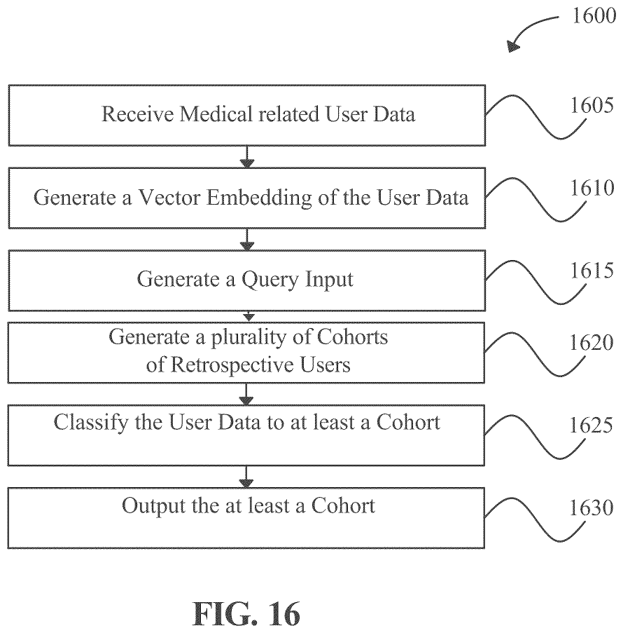

Referring now to FIG. 16, an exemplary flow diagram of a method 1600 for classifying a user to a cohort of retrospective users is illustrated. At step 1605, method 1600 includes receiving medical related user data of a user. This may be implemented as disclosed in and with reference to FIGS. 1-15. At step 1610, method 1600 includes generating a vector embedding of the user data. This may be implemented as disclosed in and with reference to FIGS. 1-15. At step 1615, method 1600 includes generating a query input. This may be implemented as disclosed in and with reference to FIGS. 1-15. At step 1620, method 1600 includes generating a plurality of cohorts of retrospective users using data extracted from a cohort database, wherein generating the plurality of cohorts comprises generating a vector embedding of an AI (Artificial Intelligence) generated query criteria to generate a cohort of retrospective users based on the query input. This may be implemented as disclosed in and with reference to FIGS. 1-15. At step 1625, method 1600 includes classifying, based on the query input, the user data to at least a cohort of the plurality of cohorts of the retrospective users. This may be implemented as disclosed in and with reference to FIGS. 1-15. At step 1630, method 1600 includes outputting the at least a cohort through a user interface. This may be implemented as disclosed in and with reference to FIGS. 1-15.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

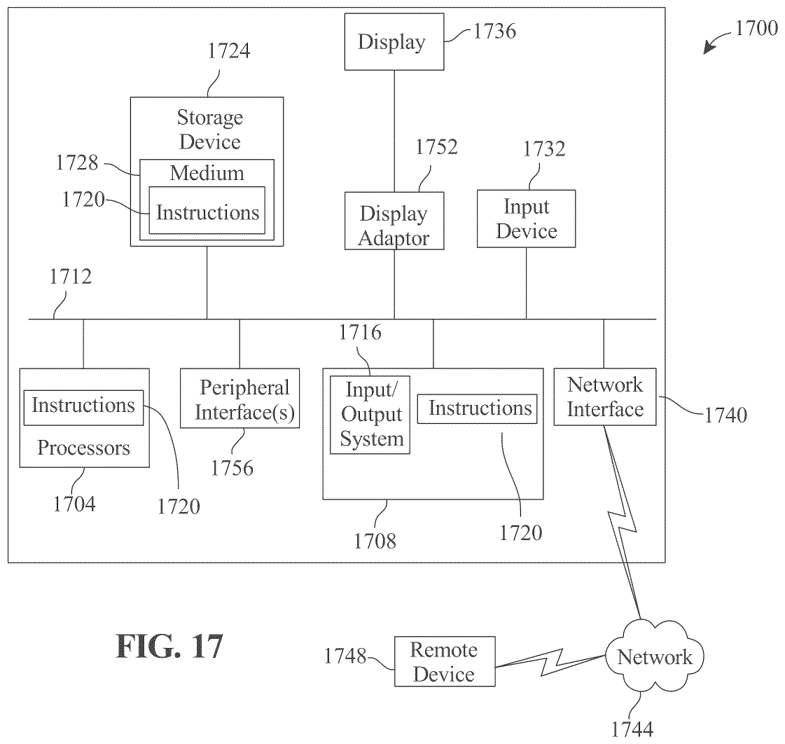

FIG. 17 shows a diagrammatic representation of one embodiment of a computing device in the exemplary form of a computer system 1700 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1700 includes a processor 1704 and a memory 1708 that communicate with each other, and with other components, via a bus 1712. Bus 1712 may include any of several types of bus structures including, but not limited to, a memory bus, a memory controller, a peripheral bus, a local bus, and any combinations thereof, using any of a variety of bus architectures.

Processor 1704 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1704 may be organized according to Von Neumann and/or Harvard architecture as a non-limiting example. Processor 1704 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), system on module (SOM), and/or system on a chip (SoC).

Memory 1708 may include various components (e.g., machine-readable media) including, but not limited to, a random-access memory component, a read only component, and any combinations thereof. In one example, a basic input/output system 1716 (BIOS), including basic routines that help to transfer information between elements within computer system 1700, such as during start-up, may be stored in memory 1708. Memory 1708 may also include (e.g., stored on one or more machine-readable media) instructions (e.g., software) 1720 embodying any one or more of the aspects and/or methodologies of the present disclosure. In another example, memory 1708 may further include any number of program modules including, but not limited to, an operating system, one or more application programs, other program modules, program data, and any combinations thereof.

Computer system 1700 may also include a storage device 1724. Examples of a storage device (e.g., storage device 1724) include, but are not limited to, a hard disk drive, a magnetic disk drive, an optical disc drive in combination with an optical medium, a solid-state memory device, and any combinations thereof. Storage device 1724 may be connected to bus 1712 by an appropriate interface (not shown). Example interfaces include, but are not limited to, SCSI, advanced technology attachment (ATA), serial ATA, universal serial bus (USB), IEEE 1364 (FIREWIRE), and any combinations thereof. In one example, storage device 1724 (or one or more components thereof) may be removably interfaced with computer system 1700 (e.g., via an external port connector (not shown)). Particularly, storage device 1724 and an associated machine-readable medium 1728 may provide nonvolatile and/or volatile storage of machine-readable instructions, data structures, program modules, and/or other data for computer system 1700. In one example, software 1720 may reside, completely or partially, within machine-readable medium 1728. In another example, software 1720 may reside, completely or partially, within processor 1704.

Computer system 1700 may also include an input device 1732. In one example, a user of computer system 1700 may enter commands and/or other information into computer system 1700 via input device 1732. Examples of an input device 1732 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1732 may be interfaced to bus 1712 via any of a variety of interfaces (not shown) including, but not limited to, a serial interface, a parallel interface, a game port, a USB interface, a FIREWIRE interface, a direct interface to bus 1712, and any combinations thereof. Input device 1732 may include a touch screen interface that may be a part of or separate from display 1736, discussed further below. Input device 1732 may be utilized as a user selection device for selecting one or more graphical representations in a graphical interface as described above.

A user may also input commands and/or other information to computer system 1700 via storage device 1724 (e.g., a removable disk drive, a flash drive, etc.) and/or network interface device 1740. A network interface device, such as network interface device 1740, may be utilized for connecting computer system 1700 to one or more of a variety of networks, such as network 1744, and one or more remote devices 1748 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1744, may employ a wired and/or a wireless mode of communication. In general, any network topology may be used. Information (e.g., data, software 1720, etc.) may be communicated to and/or from computer system 1700 via network interface device 1740.

Computer system 1700 may further include a video display adapter 1752 for communicating a displayable image to a display device, such as display device 1736. Examples of a display device include, but are not limited to, a liquid crystal display (LCD), a cathode ray tube (CRT), a plasma display, a light emitting diode (LED) display, and any combinations thereof. Display adapter 1752 and display device 1736 may be utilized in combination with processor 1704 to provide graphical representations of aspects of the present disclosure. In addition to a display device, computer system 1700 may include one or more other peripheral output devices including, but not limited to, an audio speaker, a printer, and any combinations thereof. Such peripheral output devices may be connected to bus 1712 via a peripheral interface 1756. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, apparatuses, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. An apparatus for classifying a user to a cohort of retrospective users, wherein the apparatus comprises:
at least a processor;
a computer-readable storage medium communicatively connected to the at least a processor, wherein the computer-readable storage medium contains instructions configuring the at least a processor to:
receive a query identifying a plurality of modalities of data, wherein each modality of the plurality of modalities of data represents data captured using a distinct diagnostic process;
retrieve user data of a user, wherein the user data comprises medical data corresponding to each modality of the plurality of modalities of data;
instantiate a neural network architecture, wherein instantiating the neural network architecture comprises:
instantiating, based on the query, a plurality of neural network modules configured to output a plurality modality-specific embeddings, wherein each neural network module of the plurality of neural network modules is configured to input data in modality of the plurality of modalities and output a modality-specific embedding; and
connecting the output of each of the plurality of neural network modules to at least a contrastive loss module configured to input the plurality of modality-specific embeddings and output a vector embedding;
generate a vector embedding of the user data using the neural network architecture and the user data, wherein generating the vector embedding comprises:
inputting at least a portion of the user data into at least one neural network module of the plurality of neural network modules; and
generating the vector embedding using the at least one neural network module to reduce dimensionality of the inputted at least a portion of the user data to create the vector embedding having a compact vector representation;
generate a plurality of cohorts of retrospective users using cohort data extracted from a cohort database based on a query input, wherein generating the plurality of cohorts comprises generating a set of vector embeddings of the cohort data using the neural network architecture and the cohort data;
classify, based on the vector embedding, having reduced dimensionality and compact vector representation, and the set of vector embeddings, the user data to at least a cohort of the plurality of cohorts of the retrospective users; and
output the at least a cohort through a user interface.

2. The apparatus of claim 1, wherein generating the plurality of cohorts of retrospective users further comprises:
generating a plurality of preliminary cohorts as a function of populating the cohort database; and
classifying the user to corresponding preliminary cohorts of the plurality of preliminary cohorts using a preliminary classifier.

3. The apparatus of claim 2, wherein using the preliminary classifier comprises:
training the preliminary classifier with training data comprising a plurality of user data correlated to a plurality of preliminary cohorts; and
outputting the corresponding preliminary cohorts classified to the user data.

4. The apparatus of claim 2, wherein generating the plurality of cohorts of retrospective users further comprises modifying the at least a cohort based on an intersection of the preliminary cohorts.

5. The apparatus of claim 1, wherein the at least a processor is further configured to extract biomarkers of user data to implement in a query criteria-based search of the cohort database.

6. The apparatus of claim 5, wherein extracting the biomarkers comprises implementing a machine-learning model to conduct a temporal analysis on time-series data of the user data.

7. The apparatus of claim 5, wherein extracting the biomarkers comprises implementing a machine-learning model to create measurements of biomarkers related to a plurality of biological structures of the user.

8. The apparatus of claim 1, wherein the query input comprises a criterium comprising a modality.

9. The apparatus of claim 1, wherein the at least a cohort comprises a plurality of comorbidities.

10. The apparatus of claim 9, wherein the computer-readable storage medium contains instructions further configuring the at least a processor to calculate a performance, comprising an AUC value, of a classification model on each of the plurality of comorbidities.

11. A method for classifying a user to a cohort of retrospective users, wherein the method comprises:
receive a query identifying a plurality of modalities of data, wherein each modality of the plurality of modalities of data represents data captured using a distinct diagnostic process;
retrieving, by a computing device, user data of a user, wherein the user data comprises medical data corresponding to each modality of the plurality of modalities of data;
instantiating a neural network architecture, wherein instantiating the neural network architecture comprises:
instantiating, based on the query, a plurality of neural network modules configured to output a plurality modality-specific embeddings, wherein each neural network module of the plurality of neural network modules is configured to input data in modality of the plurality of modalities and output a modality-specific embedding; and
connecting the output of each of the plurality of neural network modules to at least a contrastive loss module configured to input the plurality of modality-specific embeddings and output a vector embedding;
generating, by the computing device, a vector embedding of the user data using the neural network architecture and the user data, wherein generating the vector embedding comprises:
inputting at least a portion of the user data into at least one neural network module of the plurality of neural network modules; and
generating the vector embedding using the at least one neural network module to reduce dimensionality of the inputted at least a portion of the user data to create the vector embedding having a compact vector representation;
generating, by the computing device, a plurality of cohorts of retrospective users using cohort data extracted from a cohort database based on a query input, wherein generating the plurality of cohorts comprises generating a set of vector embeddings of the cohort data using the neural network architecture and the cohort data;
classifying, by the computing device, based on the vector embedding, having reduced dimensionality and compact vector representation, and the set of vector embeddings, the user data to at least a cohort of the plurality of cohorts of the retrospective users; and
outputting, by the computing device, the at least a cohort through a user interface.

12. The method of claim 11, wherein generating the plurality of cohorts of retrospective users further comprises:
generating a plurality of preliminary cohorts as a function of populating the cohort database; and
classifying the user to corresponding preliminary cohorts of the plurality of preliminary cohort using a preliminary classifier.

13. The method of claim 12, wherein using the preliminary classifier comprises:
training the preliminary classifier with training data comprising a plurality of user data correlated to a plurality of preliminary cohorts; and
outputting the corresponding preliminary cohorts classified to the user data.

14. The method of claim 12, wherein generating the plurality of cohorts of retrospective users further comprises modifying the at least a cohort based on an intersection of the preliminary cohorts.

15. The method of claim 11, wherein the computing device is further configured to extract biomarkers of user data to implement in a query criteria based search of the cohort database.

16. The method of claim 15, wherein extracting the biomarkers comprises implementing a machine-learning model to conduct a temporal analysis on time-series data of the user data.

17. The method of claim 15, wherein extracting the biomarkers comprises implementing a machine-learning model to create measurements of biomarkers related to a plurality of biological structures of the user.

18. The method of claim 11, wherein the query input comprises a criterium comprising a modality.

19. The method of claim 11, wherein the at least a cohort comprises a plurality of comorbidities.

20. The method of claim 19, further comprising, by the computing device, calculating a performance, comprising an AUC value, of a classification model on each of the plurality of comorbidities.
